(19) United States
(12) Patent Application Publication
(10) Pub. No.: US 2025/0035572 A1
(43) Pub. Date: Jan. 30, 2025

(54) GENERALIZATION OF THERMODYNAMIC LANGMUIR ISOTHERMS FOR MIXED-GAS ADSORPTION EQUILIBRIA

(71) Applicant: Texas Tech University System, Lubbock, TX (US)

(72) Inventors: Chau-Chyun Chen, Lubbock, TX (US); Usman Hamid, Lubbock, TX (US); Pradeep Vyawahare, Downers Grove, IL (US)

(21) Appl. No.: 18/291,981

(22) PCT Filed: Jul. 22, 2022
(86) PCT No.: PCT/US2022/037972

§ 371 Date: Jan. 25, 2024

Related U.S. Application Data
(60) Provisional application No. 63/226,303, filed on Jul. 28, 2021.

(51) Int. Cl. G01N 25/48; G06F 17/11

(52) U.S. Cl. CPC G01N 25/4813; G01N 25/4873; G06F 17/11

(57) ABSTRACT

A system and method for estimating an adsorption equilibria for one or more gases from pure component adsorption isotherms includes providing one or more processors, a memory communicably coupled to the one or more processors and an output device communicably coupled to the one or more processors, calculating an adsorption of each gas on a constant monolayer adsorption surface is calculated using the one or more processors and the generalized Langmuir isotherm equations (24)-(26) or equation (27), providing the adsorption of each gas to the output device, and developing a chemical process or product is developed using the adsorption of each gas.

### CROSS-REFERENCE TO RELATED APPLICATIONS

This application claims priority to U.S. Provisional Application Ser. No. 63/226,303, filed Jul. 28, 2021, the entire contents of which are incorporated herein by reference.

This application is related to PCT Application Serial No. PCT/US20/45586, filed Aug. 10, 2020, which claims priority to U.S. Provisional Application Ser. No. 62/860,319, filed Jun. 12, 2019. The entire contents of the foregoing applications are incorporated herein by reference.

### STATEMENT OF FEDERALLY FUNDED RESEARCH

This invention was made with government support under DE-EE0007888 awarded by the U.S. Department of Energy. The government has certain rights in the invention.

### TECHNICAL FIELD OF THE INVENTION

The present invention relates in general to the field of thermodynamic modeling, and more particularly, to a generalization of thermodynamic Langmuir isotherms for mixed-gas adsorption equilibria.

### BACKGROUND OF THE INVENTION

Without limiting the scope of the invention, its background is described in connection with Langmuir isotherm modeling.

The chemical process industry is among the most essential manufacturing sectors around the globe. The United States alone has a chemical product output of greater than $979 billion in 2019. [1] Over the years, the energy demands to operate these chemical processes grow rapidly due to an increase in chemical products demand. The separation of chemical products consumes nearly 45-55% of the industrial energy and 10-15% of the overall energy produced in the United States. [2, 3] To date, distillation, membrane-based separation, and adsorptive separation are among the widely used separation technologies. [4] However, after decades of optimization and improvements, distillation processes remain the dominant separation technologies and account for more than 50% of overall separation energy consumed due to low energy efficiency. [4, 5] Membrane-based separation is an emerging and energy-efficient technology but its industrial applications continue to be limited by low selectivity, low capacity, low stability, and elevated fouling tendency. [4, 6] In contrast, adsorptive separation is relatively established, energy-efficient, and heavily practiced in the air separation industry and others. [7]

In spite of the high industrial potential of adsorptive separation, lack of experimental measurement of mixed-gas adsorption equilibrium data hampers its wide application in industry. [8, 9] Therefore, it is imperative that robust and accurate thermodynamic models be developed and validated to reliably extrapolate and predict mixed-gas adsorption equilibria from pure component adsorption isotherms. [10] Among a wide range of models and theories proposed to estimate mixed-gas adsorption equilibria are Vacancy Solution Theory (VST) [11], Loading Ratio Correlation (LRC) [12], the extended Langmuir (eL) [13], Ideal Adsorbed Solution Theory (IAST) [14], etc. Although VST predicts azeotropic adsorption behavior, it is thermodynamically inconsistent and the model parameters lack physical significance. [7,15] The LRC model can reasonably predict mixed-gas adsorption equilibria for ideal systems. [16] However, its empiricism and failure to predict adsorption azeotrope diminish LRC's applicability to model and design adsorbers. [7, 17] Similarly, the widely practiced eL model is thermodynamically inconsistent and fails to address the pressure dependence as it presents a constant selectivity at all pressures for a given mixed-gas adsorption system. [7, 18, 19] The benchmark to predict mixed-gas adsorption equilibria from pure component adsorption isotherms and the only thermodynamically consistent model is IAST. However, IAST does not account for surface heterogeneity and it fails to predict nonideal mixed-gas adsorption equilibria. [10. 20] In short, the state-of-the-art adsorption isotherm models are either thermodynamically inconsistent or incapable of describing the nonideal behavior of mixed-gas adsorption equilibria.

Recently, for the first time, Kaur et al. [21] proposed an adsorption Nonrandom Two-Liquid (aNRTL) activity coefficient model to account for the adsorbent surface heterogeneity and its underlying adsorbate-adsorbent interactions in the adsorbate phase. Subsequently, substituting adsorbate and vacant site concentrations in the classical Langmuir (cL) adsorption rate formulation with activities using aNRTL model, Chang et al. [22] presented a thermodynamic Langmuir (tL) isotherm to capture the adsorbent surface heterogeneity for pure component adsorption and isosteric heat of adsorption. [23] Moreover, Tun and Chen [10] implemented IAST in combination with the aNRTL model and showed the feasibility to reliably correlate and predict binary and multicomponent gas adsorption equilibria from pure component isotherms at constant spreading pressure. However, the adsorbate phase activity coefficients in the IAST thermodynamic framework are functions of spreading pressure. [24] As spreading pressure is not a measurable property, it is believed that no theoretical investigations have adequately addressed the spreading pressure dependence [25] for the adsorbate phase activity coefficients.

What is needed is a new Langmuir model that allows an accurate account of both surface loading dependence and adsorbate phase composition dependence for mixed-gas adsorption equilibria.

### SUMMARY OF THE INVENTION

A simple and yet thermodynamically consistent multicomponent adsorption isotherm expression to estimate mixed-gas adsorption equilibria from pure component adsorption isotherms is described herein. The generalized Langmuir (gL) adsorption isotherm treats the adsorbent as an integral part of the adsorption system, assumes competitive adsorption of multiple adsorbates on adsorbent surface, tracks the available adsorbent vacant site surface area, and computes adsorbate and vacant site activities with an area-based aNRTL activity coefficient model. Devoid of the needs to consider the system spreading pressure constraint and the spreading pressure dependence of the adsorbate phase activity coefficients as imposed by IAST, the gL model allows an accurate account of both surface loading dependence and adsorbate phase composition dependence for mixed-gas adsorption equilibria.

In one embodiment, a computerized method for estimating an adsorption equilibria for one or more gases from pure component adsorption isotherms include providing one or more processors, a memory communicably coupled to the one or more processors and an output device communicably coupled to the one or more processors, calculating, using the one or more processors, an adsorption of each gas on a constant monolayer adsorption surface and the generalized Langmuir isotherm equations:

$${θ}_{i} = \frac{{n}_{i} {A}_{i}}{{A}^{o}} = \frac{{K}_{i}^{o} {y}_{i} P}{\frac{{γ}_{i}}{{γ}_{ϕ} {q}_{i}} + {\sum }_{j = 1}^{n} \frac{{γ}_{i} {q}_{j}}{{γ}_{j} {q}_{i}} {K}_{j}^{o} {y}_{j} P}$$

$${θ}_{ϕ} = \frac{{n}_{ϕ} {A}_{ϕ}}{{A}^{o}} = \frac{1}{1 + {\sum }_{j = 1}^{n} \frac{{γ}_{ϕ} {q}_{j}}{{γ}_{j}} {K}_{j}^{o} {y}_{j} P}$$

$${q}_{i} = \frac{{A}_{i}}{{A}_{ϕ}}$$

where: θᵢ is an adsorbate phase area fraction covered with the gas i,
nᵢAᵢ is an occupied area for the gas i,
Kᵢ^{o} is an intrinsic adsorption equilibrium constant of the gas i,
yᵢ is a gas phase mole fraction of gas i,
P is a gas vapor pressure,
γᵢ is an activity coefficient of the gas i,
γ_{ϕ} is an activity coefficient of vacant sites,
qᵢ is a ratio of an effective area of the gas i (Aᵢ) and an effective area of a phantom molecule ϕ (A_{ϕ}),
n is a number of the one or more gases,
θ_{ϕ} is an adsorbate phase vacant site area fraction, and
n_{ϕ}A_{ϕ} is a vacant area for the phantom molecule ϕ.

The adsorption of each gas is provided to the output device, and a chemical process or product is developed using the adsorption of each gas. The method can be implemented by an apparatus, system, computer, or non-transitory computer readable medium encoded with a computer program for execution by a processor that performs the steps of the method.

In one aspect, the generalized Langmuir isotherm equations reduce to

$$\frac{{n}_{i}}{{n}_{i}^{0}} = \frac{{K}_{i}^{o} {y}_{i} P}{1 + {\sum }_{i = 1}^{n} {K}_{i}^{o} {y}_{i} P}$$

when (1) the adsorbate and vacant site effective areas are the same A₁=A₂= . . . =Aᵢ=A_{ϕ}, or equivalently, the saturation loadings of adsorbates and phantom molecule are same n₁⁰=n₂⁰= . . . =n₁⁰=n_{ϕ}⁰, and (2) the adsorbate phase activity coefficients are unity γᵢ=γ_{ϕ}=1. In another aspect, the one or more gases comprise a mixed gas having two or more components. In another aspect, the constant monolayer adsorption surface comprises activated carbon, LiLSX or Zeolite H-mordenite. In another aspect, the gas i comprises CH₄, C₂H₄, C₂H₆, C₃H₆, N₂, O₂, CO₂, H₂S, or C₃H₈. In another aspect, the one or more gasses comprise a mixed gas selected from CH₄—C₂H₄, CH₄—C₂H₆, C₂H₄—C₂H₆, C₂H₄—C₃H₆, and C₂H₆—C₃H₆. In another aspect, the one or more gasses comprise a mixed gas selected from N₂ and O₂. In another aspect, the one or more gasses comprise a mixed gas selected from H₂S—CO₂, C₃H₈—H₂S, and C₃H₈—CO₂.

In another embodiment, an apparatus, system or computer includes at least one input/output interface, a data storage, and one or more processors communicably coupled to the at least one input/output interface and the data storage. The one or more processors calculate an adsorption of each gas i on a constant monolayer adsorption surface A^{o} using generalized Langmuir isotherm equations:

$${θ}_{i} = \frac{{n}_{i} {A}_{i}}{{A}^{o}} = \frac{{K}_{i}^{o} {y}_{i} P}{\frac{{γ}_{i}}{{γ}_{ϕ} {q}_{i}} + {\sum }_{j = 1}^{n} \frac{{γ}_{i} {q}_{j}}{{γ}_{j} {q}_{i}} {K}_{j}^{o} {y}_{j} P}$$

$${θ}_{ϕ} = \frac{{n}_{ϕ} {A}_{ϕ}}{{A}^{o}} = \frac{1}{1 + {\sum }_{j = 1}^{n} \frac{{γ}_{ϕ} {q}_{j}}{{γ}_{j}} {K}_{j}^{o} {y}_{j} P}$$

$${q}_{i} = \frac{{A}_{i}}{{A}_{ϕ}}$$

where: θᵢ is an adsorbate phase area fraction covered with the gas i,
nᵢAᵢ is an occupied area for the gas i,
Kᵢ^{o} is an intrinsic adsorption equilibrium constant of the gas i,
yᵢ is a gas phase mole fraction of gas i,
P is a gas vapor pressure,
γᵢ is an activity coefficient of the gas i,
γ_{ϕ} is an activity coefficient of vacant sites,
qᵢ is a ratio of an effective area of the gas i (Aᵢ) and an effective area of a phantom molecule ϕ (A_{ϕ}),
n is a number of the one or more gases, θ_{ϕ} is an adsorbate phase vacant site area fraction, and
n_{ϕ}A_{ϕ} is a vacant area for the phantom molecule ϕ.
   The adsorption of each gas i is provided to the output device, and a chemical process or a product is developed using the adsorption of each gas i.

In one aspect, the generalized Langmuir isotherm equations reduce to

$$\frac{{n}_{i}}{{n}_{i}^{0}} = \frac{{K}_{i}^{o} {y}_{i} P}{1 + {\sum }_{i = 1}^{n} {K}_{i}^{o} {y}_{i} P}$$

when (1) the adsorbate and vacant site effective areas are the same A₁=A₂= . . . =Aᵢ=A_{ϕ}, or equivalently, the saturation loadings of adsorbates and phantom molecule are same n₁⁰=n₂⁰= . . . =nᵢ⁰=n_{ϕ}⁰, and (2) the adsorbate phase activity coefficients are unity γᵢ=γ_{ϕ}=1. In another aspect, the one or more gases comprise a mixed gas having two or more components. In another aspect, the constant monolayer adsorption surface comprises activated carbon, LiLSX or Zeolite H-mordenite. In another aspect, the gas i comprises CH₄, C₂H₄, C₂H₆, C₃H₆, N₂, O₂, CO₂, H₂S, or C₃H₈. In another aspect, the one or more gasses comprise a mixed gas selected from CH₄—C₂H₄, CH₄—C₂H₆, C₂H₄—C₂H₆, C₂H₄—C₃H₆, and C₂H₆—C₂H₆. In another aspect, the one or more gasses comprise a mixed gas selected from N₂ and O₂. In another aspect, the one or more gasses comprise a mixed gas selected from H₂S—CO₂, C₃H₈—H₂S, and C₃H₈—CO₂.

In another embodiment, a method of adsorbing one or more gases includes providing a vessel containing a constant monolayer adsorption surface, introducing one or more gases into the vessel, wherein the adsorption of each gas on the constant monolayer adsorption surface is determined by the generalized Langmuir isotherm equations:

$${θ}_{i} = \frac{{n}_{i} {A}_{i}}{{A}^{o}} = \frac{{K}_{i}^{o} {y}_{i} P}{\frac{{γ}_{i}}{{γ}_{ϕ} {q}_{i}} + {\sum }_{j = 1}^{n} \frac{{γ}_{i} {q}_{j}}{{γ}_{j} {q}_{i}} {K}_{j}^{o} {y}_{j} P}$$

$${θ}_{ϕ} = \frac{{n}_{ϕ} {A}_{ϕ}}{{A}^{o}} = \frac{1}{1 + {\sum }_{j = 1}^{n} \frac{{γ}_{ϕ} {q}_{j}}{{γ}_{j}} {K}_{j}^{o} {y}_{j} P}$$

$${q}_{i} = \frac{{A}_{i}}{{A}_{ϕ}}$$

where: θᵢ is an adsorbate phase area fraction covered with the gas i,
nᵢAᵢ is an occupied area for the gas i,
Kᵢ^{o} is an intrinsic adsorption equilibrium constant of the gas i,
yᵢ is a gas phase mole fraction of gas i,
P is a gas vapor pressure,
γᵢ is an activity coefficient of the gas i,
γ_{ϕ} is an activity coefficient of vacant sites,
qᵢ is a ratio of an effective area of the gas i (Aᵢ) and an effective area of a phantom molecule ϕ (A_{ϕ}),
n is a number of the one or more gases,
θ_{ϕ} is an adsorbate phase vacant site area fraction, and
n_{ϕ}A_{ϕ} is a vacant area for the phantom molecule ϕ.

The adsorption of each gas i is provided to the output device, and a chemical process or a product is developed using the adsorption of each gas i. A product can be produced in accordance with this method.

In one aspect, the generalized Langmuir isotherm equations reduce to

$$\frac{{n}_{i}}{{n}_{i}^{0}} = \frac{{K}_{i}^{o} {y}_{i} P}{1 + {\sum }_{i = 1}^{n} {K}_{i}^{o} {y}_{i} P}$$

when (1) the adsorbate and vacant site effective areas are the same A₁=A₂= . . . =Aᵢ=A_{ϕ}, or equivalently, the saturation loadings of adsorbates and phantom molecule are same n₁⁰=n₂⁰= . . . =nᵢ⁰=n_{ϕ}⁰, and (2) the adsorbate phase activity coefficients are unity γᵢ=γ_{ϕ}=1. In another aspect, the one or more gases comprise a mixed gas having two or more components. In another aspect, the constant monolayer adsorption surface comprises activated carbon, LiLSX or Zeolite H-mordenite. In another aspect, the gas i comprises CH₄, C₂H₄, C₂H₆, C₃H₆, N₂, O₂, CO₂, H₂S, or C₃H₈. In another aspect, the one or more gasses comprise a mixed gas selected from CH₄—C₂H₄, CH₄—C₂H₆, C₂H₄—C₂H₆, C₂H₄—C₃H₆, and C₂H₆—C₃H₆. In another aspect, the one or more gasses comprise a mixed gas selected from N₂ and O₂. In another aspect, the one or more gasses comprise a mixed gas selected from H₂S—CO₂, C₃H₈—H₂S, and C₃H₈—CO₂.

### BRIEF DESCRIPTION OF THE DRAWINGS

For a more complete understanding of the features and advantages of the present invention, reference is now made to the detailed description of the invention along with the accompanying figures and in which:

FIGS. 1A to 1C depict the data sets and model results for five pure component adsorption isotherms: (FIG. 1A) CH₄, C₂H₄, C₂H₆, and C₃H₆ on activated carbon at 323 K [28], (FIG. 1B) N₂ and O₂ on LiLSX at 303.15 K [29], (FIG. 1C) CO₂, H₂S, and C₃H₈ on zeolite H-mordenite at 303.15 K [30];

FIGS. 2A to 2E depict the experimental data and model results for the five binary adsorption equilibrium compositions on activated carbon at 323 K and 0.1 bar [28]: (FIG. 2A) CH₄—C₂H₄, (FIG. 2B) CH₄—C₂H₆, (FIG. 2C) C₂H₄—C₂H₆, (FIG. 2D) C₂H₄—C₃H₆, and (FIG. 2E) C₂H₆—C₃H₆;

FIG. 3 depicts the experimental data and model results for the mixed-gas adsorption of N₂—O₂ binary mixture on LiLSX at 1.013 bar and 6.08 bar at 303.15 K [29];

FIG. 4A depicts the experimental data and the model results for mixed-gas adsorption of H₂S CO₂ binary mixture on zeolite H-mordenite at 303.15 K and 0.156 bar [30];

FIGS. 4B and 4C depicts the adsorption azeotropic behaviors of (FIG. 4B) C₃H₈—H₂S and (FIG. 4C) C₃H₈—CO₂ binary gas mixtures at 303.15 K on zeolite H-mordenite at 0.081 bar and 0.41 bar respectively [30];

FIG. 5A depicts the overall surface loading jumps of N₂—O₂ on LiLSX when the system pressure jumps from 1.013 bar to 6.08 bar at 303.15 K [29];

FIGS. 5B and 5C depict the adsorbent surface area tracking for (FIG. 5B) C₃H₈—H₂S on zeolite H-mordenite at 0.081 bar and 303.15 K [30], and (FIG. 5C) C₃H₈—CO₂ on zeolite H-mordenite at 0.41 bar and 303.15 K [30];

FIGS. 6A and 6B depict the mixed-gas adsorption equilibria phase diagram for (FIG. 6A) N₂—O₂ on LiLSX at 1.013 bar and 6.08 bar at 303.15 K [29], and (FIG. 6B) C₃H₈—H₂S at 0.081 bar and C₃H₈—CO₂ at 0.41 bar on zeolite H-mordenite at 303.15 K [30];

FIG. 7A depicts the activity coefficient of binary mixtures N₂—O₂ on LiLSX at 1.013 bar and 6.08 bar at 303.15 K [29];

FIGS. 7B and 7C depict the activity coefficient of binary mixtures (FIG. 7B) C₃H₈—H₂S on zeolite H-mordenite at 0.081 bar and 303.15 K [30], and (FIG. 7C) C₃H₈—CO₂ on zeolite H-mordenite at 0.41 bar and 303.15 K [30];

FIGS. 8A to 8C depict the ternary mixed-gas adsorption equilibria: (FIG. 8A) CH, (∘)-C₂H, (Δ)-C₂H, (x) on activated carbon at 323 K and 0.1 bar [28], (FIG. 8B) C₂H₄(∘)-C₂H, (Δ)-C₃H₆ (x) on activated carbon at 323 K and 0.1 bar [28], (FIG. 8C) CO₂ (∘)-H₂S (Δ)-C₃H₈ (x) on zeolite H-mordenite at 303.15 K and 0.007-0.1342 bar [30], where the blue, black, and red colors represent eL, IAST, and gL predictions, respectively;

FIG. 9 is a block diagram of an apparatus or system suitable for performing the methods described herein;

FIG. 10 is a flow chart of a method for estimating an adsorption equilibria for one or more gases from pure component adsorption isotherms; and

FIG. 11 is a flow chart of a method for adsorbing one or more gases.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity but include the general class of which a specific example may be used for illustration. The terminology herein is used to describe specific embodiments of the invention, but their usage does not limit the invention, except as outlined in the claims.

A thermodynamically consistent model to predict mixed-gas adsorption equilibria from pure gas adsorption isotherms is described herein. A generalization of thermodynamic Langmuir isotherm for pure component adsorption, the model assumes competitive adsorption of multiple adsorbates on adsorbent surface and it applies an area-based adsorption Nonrandom Two-Liquid activity coefficient model in the activity coefficient calculations for the adsorbate phase. The resulting generalized Langmuir (gL) isotherm properly captures both surface loading dependence and adsorbate phase composition dependence for mixed-gas adsorption equilibria. The model is validated with accurate representations of gas adsorption equilibrium data for wide varieties of unary, binary, and ternary gas systems. The model results are further compared with those calculated from extended Langmuir isotherm and Ideal Adsorbed Solution Theory.

### Model Formulation

### Generalized Langmuir Isotherm for Pure Component Adsorption

Starting from the fundamental adsorption and desorption reactions of pure adsorbate gas A on an adsorbent surface containing vacant sites S:

$${A}_{( g )} + S ↔ AS \qquad ( 1 )$$

The adsorption reaction with rate constant kₐ results in occupied sites denoted with AS. In contrast, the desorption reaction having rate constant k_{d} results in pure gas A and vacant sites S. Once the adsorption equilibrium has been achieved, the rates of adsorption and desorption become equal as shown in Eq. (2).

$${k}_{a} P [ S ] = {k}_{d} [ AS ] \qquad ( 2 )$$

Here [S] and [AS] denote the classical Langmuir (cL) site concentrations of vacant sites and occupied sites, respectively. The occupied sites can be expressed in the amount adsorbed for adsorbate component 1, n₁. The vacant sites can be represented as (n₁⁰−n₁), where n₁⁰ is the saturation amount adsorbed for component 1. The apparent adsorption equilibrium constant K₁ can be expressed as a function of the adsorbate phase site concentrations as:

$${K}_{1} = \frac{{k}_{o}}{{k}_{d}} = \frac{[ AS ]}{P [ S ]} = \frac{{n}_{1}}{P ( {n}_{1}^{0} - {n}_{1} )} = \frac{{x}_{1}}{P ( 1 - {x}_{1} )} \qquad ( 3 )$$

where x₁ is the ratio of n₁ and n₁⁰. Simplification of Eq. (3) for x₁ yields the classical Langmuir isotherm shown in Eq. (4):

$$\frac{{n}_{1}}{{n}_{1}^{0}} = \frac{{K}_{1} P}{1 + {K}_{1} P} \qquad ( 4 )$$

The classical Langmuir isotherm disregards the adsorbent surface heterogeneity upon assuming the rates of adsorption and desorption are proportional to the site concentrations. Chang et al. [22] proposed the thermodynamic Langmuir isotherm to address the surface heterogeneity by substituting the site concentrations with the site activities expressed as the product of site concentration and site activity coefficient as shown in Eq. (5), leading to the thermodynamic Langmuir isotherm expressed as Eq. (6):

$${K}_{1}^{o} = \frac{{x}_{1} {γ}_{1}}{{γ}_{ϕ} {x}_{ϕ} P} \qquad ( 5 )$$

$$\frac{{n}_{1}}{{n}_{1}^{0}} = \frac{{K}_{1}^{o} P}{\frac{{γ}_{1}}{{γ}_{ϕ}} + {K}_{1}^{o} P} \qquad ( 6 )$$

where K₁^{o} is the intrinsic adsorption equilibrium constant of adsorbate component 1, x₁ is the ratio of n₁ and n₁⁰, and x_{ϕ}, representing the vacant site fraction on the adsorbent surface, is calculated as (1−x₁). y₁ is the activity coefficient of component 1 on the occupied sites and γ_{ϕ} is the activity coefficient of “phantom” molecule ϕ on the vacant sites. The reference state for adsorbate component 1 is the adsorbate phase fully occupied with component 1 while the reference state for the phantom molecule ϕ is the absorbate phase with vacant sites only.

In addition to addressing the surface heterogeneity, this work further proposes that there is a constant total adsorbent surface area, A^{o}, which is covered with adsorbate component 1 with the “effective” molecular area, A₁. The adsorbate phase area fraction covered with component 1, θ₁, is a function of A^{o}, A₁, and n₁, expressed as:

$${n}_{T} = {n}_{1} + {n}_{ϕ} \qquad ( 7 )$$

$${x}_{1} = \frac{{n}_{1}}{{n}_{T}} \qquad ( 8 )$$

$${x}_{ϕ} = \frac{{n}_{ϕ}}{{n}_{T}} \qquad ( 9 )$$

$${θ}_{1} = \frac{{n}_{1} {A}_{1}}{{A}^{o}} \qquad ( 10 )$$

$${θ}_{ϕ} = \frac{{n}_{ϕ} {A}_{ϕ}}{{A}^{o}} \qquad ( 11 )$$

$${A}^{o} = {n}_{1} {A}_{1} + {n}_{ϕ} {A}_{ϕ} = {n}_{1}^{0} {A}_{1} = {n}_{ϕ}^{0} {A}_{ϕ} \qquad ( 12 )$$

where n_{ϕ} and n_{ϕ}⁰ are the remaining amount and the maximum amount of vacant sites, respectively, A_{ϕ} is the “effective” area of phantom molecule ϕ, with nitrogen chosen as the model molecule for ϕ. x₁ is the ratio of n₁ and n_{T} referred as the adsorbate phase mole fraction of component 1, and x_{ϕ} is the ratio of n_{ϕ} and n_{T} and referred as the adsorbate phase mole fraction of vacant sites. While A^{o} remains constant, the occupied site area fraction, θ₁, and the vacant site area fraction, θ_{ϕ}, change with respect to the loading of component 1. Upon considering both the site activities and the site area fractions, the generalized Langmuir isotherm is expressed in Eqs. (13)-(14) for adsorbate component 1 and vacant site area fractions:

$${θ}_{1} = \frac{{n}_{1} {A}_{1}}{{A}^{o}} = \frac{{K}_{1}^{o} P}{\frac{{γ}_{1}}{{γ}_{ϕ} {q}_{1}} + {K}_{1}^{o} P} \qquad ( 13 )$$

$${θ}_{ϕ} = \frac{{n}_{ϕ} {A}_{ϕ}}{{A}^{o}} = \frac{1}{1 + \frac{{γ}_{ϕ} {q}_{1}}{{γ}_{1}} {K}_{1}^{o} P} \qquad ( 14 )$$

$${q}_{1} = \frac{{A}_{1}}{{A}_{ϕ}} \qquad ( 15 )$$

Here, q₁ is the ratio of effective area of component 1 and the effective area of phantom molecule ϕ and it should depend on the adsorbent surface characteristics, adsorption temperature, and the local minimal energy adsorbate molecular configuration on the adsorbent surface. [26] γ₁ and γ_{ϕ} are the activity coefficients of component 1 and phantom molecule, respectively, which are functions of x₁, x_{ϕ}, A₁, and A_{ϕ}. Eq. (13) reduces to the thermodynamic Langmuir (tL) isotherm, Eq. (6), when the effective area of adsorbate is same as the effective area of phantom molecule. Eq. (13) further reduces to the classical Langmuir isotherm, Eq. (4), when both the effective area of adsorbate and the effective area of phantom molecule are same and the activity coefficients are unity.

### Generalized Langmuir Isotherm for Mixed-Gas Adsorption

The generalized Langmuir isotherm for pure component adsorption can be readily extended for mixed-gas adsorption. For a competitive adsorption of multiple adsorbates on a constant monolayer adsorption surface area, A°, the corresponding generalized Langmuir isotherm equations for mixed-gas adsorption equilibria can be expressed as:

$${K}_{i}^{o} = \frac{{x}_{i} {γ}_{i}}{{γ}_{ϕ} {x}_{ϕ} {Py}_{i}} \qquad ( 16 )$$

$${n}_{T} = {\sum }_{i = 1}^{n} {n}_{i} + {n}_{ϕ} \qquad ( 17 )$$

$${x}_{i} = \frac{{n}_{i}}{{n}_{T}} \qquad ( 18 )$$

$${x}_{ϕ} = \frac{{n}_{ϕ}}{{n}_{T}} \qquad ( 19 )$$

$${\sum }_{i = 1}^{n} {x}_{i} + {x}_{ϕ} = 1 \qquad ( 20 )$$

$${θ}_{i} = \frac{{n}_{i} {A}_{i}}{{A}^{o}} \qquad ( 21 )$$

$${θ}_{ϕ} = \frac{{n}_{ϕ} {A}_{ϕ}}{{A}^{o}} \qquad ( 22 )$$

$${A}^{o} = {\sum }_{i = 1}^{n} {n}_{i} {A}_{i} + {n}_{ϕ} {A}_{ϕ} = {n}_{i}^{0} {A}_{i} = {n}_{ϕ}^{0} {A}_{ϕ} \qquad ( 23 )$$

where γᵢ is the gas phase mole fraction of adsorbate component i, n is the number of adsorbates, n_{T} is the sum of the total amount adsorbed of all adsorbates and the remaining amount of vacant sites. θᵢ is the adsorbate phase area fraction covered with component i, and θ_{ϕ} is the adsorbate phase vacant site area fraction. Eq. (16) defines the intrinsic adsorption equilibrium constant of component i while Eqs. (17)-(20) express the adsorbate phase mole fractions of component i and vacant sites and their relationships. Eqs. (21) and (22) track the adsorbate phase area fractions occupied by adsorbate component i and vacant sites, respectively. Eq. (23) shows, while the total adsorbent surface area, A^{o}, remains constant, the occupied area for component i, nᵢAᵢ and the are for vacant sites, n_{ϕ}A_{ϕ}, vary with loading and adsorbate phase compositions.

Rearrangement of Eqs. (16)-(20) in combination with Eqs. (21)-(22) results in the generalized Langmuir isotherm equations for mixed-gas adsorption equilibria as presented in Eqs. (24) and (25).

$${θ}_{i} = \frac{{n}_{1} {A}_{1}}{{A}^{o}} = \frac{{K}_{i}^{o} {y}_{i} P}{\frac{{γ}_{i}}{{γ}_{ϕ} {q}_{i}} + {\sum }_{{j = 1}^{}}^{n} \frac{{γ}_{i} {q}_{j}}{{γ}_{j} {q}_{i}} {K}_{j}^{o} {y}_{j} P} \qquad ( 24 )$$

$${θ}_{ϕ} = \frac{{n}_{ϕ} {A}_{ϕ}}{{A}^{o}} = \frac{1}{1 + {\sum }_{{j = 1}^{}}^{n} \frac{{γ}_{ϕ} {q}_{j}}{{γ}_{j}} {K}_{j}^{o} {y}_{j} P} \qquad ( 25 )$$

$${q}_{i} = \frac{{A}_{i}}{{A}_{ϕ}} \qquad ( 26 )$$

Here, qᵢ is the ratio of effective area of component i and effective area of phantom molecule, γᵢ and γ_{ϕ} represent the activity coefficients of component i and vacant sites, respectively, which are functions of xᵢ's, x_{ϕ}, Aᵢ's, and A_{ϕ}. In summary, solving n number of Eq. (24) for n number of adsorbates coupled with Eq. (25) for n_{ϕ} along with an appropriate activity coefficient model results in the generalized Langmuir isotherm for mixed-gas adsorption equilibria. The generalized Langmuir isotherm reduces to the extended Langmuir isotherm, shown in Eq. (27), if the following two conditions are satisfied: 1) the adsorbate and vacant site effective areas are the same, i.e., A₁=A₂= . . . =Aᵢ=A_{ϕ}, or equivalently, the saturation loadings of adsorbates and phantom molecule are same, i.e., n₁⁰=n₂⁰= . . . =nᵢ=n₀⁰, and 2) the adsorbate phase activity coefficients are unity. Devoid of the simplifying assumptions of the extended Langmuir isotherm, the generalized Langmuir isotherm provides a thermodynamically consistent generalization of the extended Langmuir expression.

$$\frac{{n}_{i}}{{n}_{i}^{0}} = \frac{{K}_{i}^{o} {y}_{i} P}{1 + {\sum }_{i = 1}^{n} {K}_{i}^{o} {y}_{i} P} \qquad ( 27 )$$

Area-Based Multicomponent aNRTL Model

The original adsorption NRTL model derived from the two-fluid theory does not take into account the effective areas of adsorbates and phantom molecule on the adsorbate phase. [21] To make aNRTL model consistent with the generalized Langmuir model formulation, the aNRTL model formulation has been revisited to incorporate the adsorbate area fractions. The derivation of area-based aNRTL model is provided below.

Consider an adsorbent surface containing energetically non-interacting multiple adsorption sites where each site “0” is surrounded with the molecules of n number of adsorbates. To address the local composition of multicomponent adsorbate phase, Nonrandom two-liquid theory [36] has been implied while incorporating the size of adsorbates. So, the sum of local area fractions of n adsorbates around the adsorbent site “0” shall be unity, written as:

$${\sum }_{i = 1}^{n} {θ}_{i 0} = 1 \qquad ( 28 )$$

Here, θᵢ₀ is the local area fractions of ith adsorbate near the adsorbent site “0” that can be expressed in the form of area fraction and mole fraction as follow:

$${θ}_{i 0} = \frac{{θ}_{i} exp ( - \frac{α {g}_{i 0}}{RT} )}{{\sum }_{k = 1}^{n} {θ}_{k} exp ( - \frac{α {g}_{k 0}}{RT} )} = \frac{{θ}_{i} exp ( - α \frac{{g}_{i 0} - {g}_{00}}{RT} )}{{\sum }_{k = 1}^{n} {θ}_{k} exp ( - α \frac{{g}_{k 0} - {g}_{00}}{RT} )} = \frac{{θ}_{i} {G}_{i 0}}{{\sum }_{k = 1}^{n} {θ}_{k} {G}_{k 0}} \qquad ( 29 )$$

$${θ}_{i} = \frac{{x}_{i} {q}_{i}}{{\sum }_{i = 1}^{n} {x}_{i} {q}_{i}} \qquad ( 30 )$$

Here, gᵢ indicates the interaction energy between the ith adsorbate and the adsorption site. α denotes the non-randomness factor and qᵢ is the effective area of the ith adsorbate.

To derive the expression for molar excess Gibbs energy for area based aNRTL model, the multicomponent adsorbate system is treated with the two-liquid theory. [37] Starting from the sum of changes of residual Gibbs energy of the adsorbate phase:

$${g}^{mix} = {\sum }_{j = 1}^{n} = {x}_{j} {q}_{j} {g}^{( r )} \qquad ( 31 )$$

$${g}^{E} = {\sum }_{j = 1}^{n} {x}_{j} {q}_{j} ( {\sum }_{i = 1}^{n} {θ}_{i 0} {g}_{i 0} - {g}_{j 0} ) \qquad ( 32 )$$

Here g is the residual Gibbs energy. Now, using Eq. (29) and (32) results in:

$${g}^{E} = {\sum }_{{j = 1}^{}}^{n} {x}_{j} {q}_{j} {\sum }_{{i = 1}^{}}^{n} {g}_{i 0} \frac{{θ}_{i} exp ( - \frac{α {g}_{i 0}}{RT} )}{{\sum }_{{k = 1}^{}}^{n} {θ}_{k} exp ( - \frac{α {g}_{k 0}}{RT} )} - {\sum }_{{j = 1}^{}}^{n} {x}_{j} {q}_{j} {g}_{j 0} \qquad ( 33 )$$

$${g}^{E} = \frac{{\sum }_{{i = 1}^{}}^{n} {x}_{i} {q}_{i} exp ( - \frac{α {g}_{i 0}}{RT} ) \cdot [ {\sum }_{{j = 1}^{}}^{n} {x}_{j} {q}_{j} {g}_{i 0} - {\sum }_{{j = 1}^{}}^{n} {x}_{j} {q}_{j} {g}_{j 0} ]}{{\sum }_{k - 1}^{n} {x}_{k} {q}_{k} exp ( - \frac{α {g}_{k 0}}{RT} )} \qquad ( 34 )$$

Eq. (34) can be simplified to Eq. (35):

$$\frac{{g}^{E}}{RT} = {\sum }_{i = 1}^{n} {x}_{i} {q}_{i} {G}_{i 0} \frac{{\sum }_{j = 1}^{n} {x}_{j} {q}_{j} [ \frac{{g}_{i 0} - {g}_{j 0}}{RT} ]}{{\sum }_{k = 1}^{n} {x}_{k} {q}_{k} {G}_{k 0}} \qquad ( 35 )$$

Simplification of Eq. (35) results in the final expression of Gibbs energy for multicomponent adsorbate systems, written as:

$$\frac{{g}^{E}}{RT} = {\sum }_{i = 1}^{n} {x}_{i} {q}_{i} \frac{{\sum }_{j = 1}^{n} {x}_{j} {q}_{j} {τ}_{ij}}{{\sum }_{k = 1}^{n} {x}_{k} {q}_{k} {G}_{k i}} \qquad ( 36 )$$

Afterwards, the activity coefficients are related to the partial molar excess Gibbs energy expressed as follow:

$$ln {γ}_{i} = \frac{\partial }{\partial {n}_{i}} {[ \frac{{n}_{T} {g}^{E}}{RT} ]}_{T , π , {n}_{j \neq i}} \qquad ( 37 )$$

Rearranging Eq. (36) and then substituting in Eq. (37) results in:

$$\frac{{n}_{T} {g}^{E}}{RT} = {\sum }_{p} {n}_{p} {q}_{p} \frac{{\sum }_{j} {n}_{j} {q}_{j} {τ}_{pj}}{{\sum }_{k} {n}_{k} {q}_{k} {G}_{kp}} \qquad ( 38 )$$

$$\frac{{n}_{T} {g}^{E}}{RT} = {\underbrace{{n}_{i} {q}_{i} \frac{{\sum }_{j} {n}_{j} {q}_{j} {τ}_{ij}}{{\sum }_{k} {n}_{k} {q}_{k} {G}_{ki}}}}_{{1}^{st} term} + {\underbrace{{\sum }_{p \neq i} {n}_{p} {q}_{p} \frac{{\sum }_{j} {n}_{j} {q}_{j} {τ}_{pj}}{{\sum }_{k} {n}_{k} {q}_{k} {G}_{kp}}}}_{{2}^{nd} term} \qquad ( 39 )$$

Here, p=j=k=1, 2, 3, . . . , i, . . . , m. Now, taking the derivative of Eq. (39) with respect to ng at constant T, π, and n_{j≠i};

$$\frac{\partial }{\partial {n}_{i}} {[ \frac{{n}_{T} {g}^{E}}{RT} ]}_{{1}^{st} term} = - {n}_{i} {q}_{i}^{2} \cdot \frac{{\sum }_{j} {n}_{j} {q}_{j} {τ}_{ij}}{{( {\sum }_{k} {n}_{k} {q}_{k} {G}_{ki} )}^{2}} + {q}_{i} \cdot \frac{{\sum }_{j} {n}_{j} {q}_{j} {τ}_{ij}}{{\sum }_{k} {n}_{k} {q}_{k} {G}_{ki}} \qquad ( 40 )$$

Upon simplification, Eq. (40) can be written as:

$$\frac{\partial }{\partial {n}_{i}} {[ \frac{{n}_{T} {g}^{E}}{RT} ]}_{{1}^{st} term} = - {n}_{i} {q}_{i}^{2} \cdot \frac{{\sum }_{j} {n}_{j} {q}_{j} {τ}_{ij}}{{( {\sum }_{k} {n}_{k} {q}_{k} {G}_{ki} )}^{2}} + {q}_{i} \cdot \frac{{\sum }_{j} {n}_{j} {q}_{j} {τ}_{ij} {G}_{ij}}{{\sum }_{k} {n}_{k} {q}_{k} {G}_{kj}} \qquad ( 41 )$$

Now, taking the partial derivative of 2^{nd} term as expressed in Eq. (41):

$$\frac{\partial }{\partial {n}_{i}} {[ \frac{{n}_{T} {g}^{E}}{RT} ]}_{{2}^{nd} term} = {\sum }_{p \neq i} {n}_{p} {q}_{p} ( \frac{{\sum }_{k} {n}_{k} {q}_{k} {G}_{kp} \cdot {q}_{i} {τ}_{pi} - {\sum }_{j} {n}_{j} {q}_{j} {τ}_{pj} \cdot {q}_{i} {G}_{ip}}{{( {\sum }_{k} {n}_{k} {q}_{k} {G}_{kp} )}^{2}} ) \qquad ( 42 )$$

Upon simplification of Eq. (42), the resulting expressions are:

$$\frac{\partial }{\partial {n}_{i}} {[ \frac{{n}_{T} {g}^{E}}{RT} ]}_{{2}^{nd} term} = {q}_{i} \frac{{\sum }_{p \neq i} [ {\sum }_{j \neq i} {n}_{p} {q}_{p} {G}_{p 0} \cdot {n}_{j} {q}_{j} ( {τ}_{pi} {G}_{j 0} - {τ}_{pj} {G}_{i 0} ) ]}{{( {\sum }_{k} {n}_{k} {q}_{k} {G}_{k 0} )}^{2}} \qquad ( 43 )$$

$$\frac{\partial }{\partial {n}_{i}} {[ \frac{{n}_{T} {g}^{E}}{RT} ]}_{{2}^{nd} term} = {q}_{i} {\sum }_{j} {n}_{j} {q}_{j} \frac{{\sum }_{p} {n}_{p} {q}_{p} {G}_{pj} ( {τ}_{pi} - {τ}_{pj} {G}_{ij} )}{{( {\sum }_{k} {n}_{k} {q}_{k} {G}_{{k}_{j}} )}^{2}} + {q}_{i} {\sum }_{j} {n}_{j} {q}_{j} \frac{{n}_{i} {q}_{i} {τ}_{ij} {G}_{ij}^{2}}{{( {\sum }_{k} {n}_{k} {q}_{k} {G}_{kj} )}^{2}} \qquad ( 44 )$$

Now, combining Eq. (41) and (44) results in:

$$ln {γ}_{i} = {q}_{i} {\sum }_{j} {n}_{j} {q}_{j} [ \frac{{τ}_{ij} {G}_{ij}}{{\sum }_{k} {n}_{k} {q}_{k} {G}_{kj}} + \frac{{\sum }_{p} {n}_{p} {q}_{p} {G}_{pj} ( {τ}_{pi} - {τ}_{pj} {G}_{ij} )}{{( {\sum }_{k} {n}_{k} {q}_{k} {G}_{kj} )}^{2}} ] \qquad ( 45 )$$

Simplification of Eq. (45) provides the final expression of activity coefficient of component i in the multicomponent gas adsorption equilibria system.

$$ln {γ}_{i} = \frac{\partial }{\partial {n}_{i}} {[ \frac{{n}_{T} {g}^{E}}{RT} ]}_{T , π , {n}_{j \neq i}} = {q}_{i} \frac{{\sum }_{j = 1}^{n} {x}_{j}^{2} {q}_{j}^{2} {τ}_{ij} [ {G}_{ij} - 1 ]}{{( {\sum }_{k = 1}^{n} {x}_{k} {q}_{k} {G}_{kj} )}^{2}} \qquad ( 46 )$$

Here, qᵢ=Aᵢ/A_{ϕ} is the ratio of effective adsorbate area of component i and that of the model molecule (nitrogen) for phantom molecule, and xᵢ is the adsorbate phase mole fraction of component i.

$${τ}_{ij} = - {τ}_{ji} = \frac{{g}_{i 0} - {g}_{j 0}}{RT} \qquad ( 47 )$$

$${G}_{ij} = \frac{1}{{G}_{ji}} = exp ( - α {τ}_{ij} ) \qquad ( 48 )$$

It is worth noting that the gL isotherm treats the adsorbent as a part of the adsorption system. In other words, the adsorbate phase of a single component gas adsorption system is treated as a binary system of adsorbate component 1 and phantom molecule ϕ for adsorbent vacant sites with the composition of (x₁, x_{ϕ}). Likewise, the adsorbate phase of a binary gas adsorption system is treated as a ternary system of two adsorbates and phantom molecule with the composition of (x₁, x₂, x_{ϕ}). For a mixed-gas adsorption system with n components, adsorbent vacant sites or phantom molecule ϕ included in the mixture, the excess Gibbs energy expression for the adsorbate phase is:

$$\frac{{g}^{E}}{RT} = {\sum }_{i = 1}^{n} {x}_{i} {q}_{i} \frac{{\sum }_{j = 1}^{n} {x}_{j} {q}_{j} {τ}_{ij}}{{\sum }_{k = 1}^{n} {x}_{k} {q}_{k} {G}_{ki}} \qquad ( 49 )$$

The corresponding activity coefficient expression is given in Eq. (50):

$$ln {γ}_{i} = {q}_{i} \frac{{{\sum }_{j = 1}}^{n} {x}_{j}^{2} {q}_{j}^{2} {τ}_{ij} [ {G}_{ij} - 1 ]}{{( {\sum }_{k = 1}^{n} {x}_{k} {q}_{k} {G}_{kj} )}^{2}} \qquad ( 50 )$$

$${τ}_{ij} = - {τ}_{ji} = \frac{{g}_{i 0} - {g}_{j 0}}{RT} \qquad ( 51 )$$

$${G}_{ij} = exp ( - {ατ}_{ij} ) \qquad ( 52 )$$

where α is the nonrandomness factor fixed at 0.3 and τᵢⱼ's are the adjustable binary interaction parameters for the i-j pair, with τᵢⱼ=−τⱼᵢ. In short, the area-based adsorption NRTL activity coefficients are functions of xᵢ's, x_{ϕ}, Aᵢ's, and A_{ϕ}, and τᵢⱼ's.

### Results and Discussion

### Pure Component Adsorption Isotherm

Accurate representation of experimental pure component adsorption isotherm data is the first step towards successful modeling of mixed-gas adsorption equilibria. The classical Langmuir (cL), thermodynamic Langmuir (tL), and generalized Langmuir (gL) models are used herein to represent pure component adsorption isotherms. Requiring no activity coefficient calculations, cL makes use of two model parameters (Kᵢ and nᵢ⁰). In contrast, both tL and gL isotherms require three model parameters (τ_{iϕ}, Kᵢ^{o}, and nᵢ⁰). For internal consistency, the original aNRTL model [21] is used in the activity coefficient calculation for tL and the area-based aNRTL model presented above is used with gL. Note that, with literature reported values for A^{o} and Aᵢ the nᵢ⁰ parameter in gL can be calculated per Eq. (23) and leave gL with only two adjustable model parameters, i.e., τ_{iϕ} and Kᵢ^{o}. The effective areas in gL, Aᵢ's, are notably dependent on adsorbent, adsorption temperature, and the configuration of adsorbate molecule on the adsorbent surface. [26] The recommended effective areas of adsorbates and adsorbent surface areas are available in the literature. [26] To regress the adsorption isotherm parameters, a Maximum Likelihood Principle-based objective function is minimized, [27] given as follow:

$$Obj = {\sum }_{i} {( \frac{{n}_{i}^{calc} - {n}_{i}^{expt}}{{σ}^{expt}} )}^{2} \qquad ( 53 )$$

where σ indicates the standard deviation of the experimental data set to 0.05 mol/kg while nᵢ^{calc} and nᵢ^{expt} denote the calculated and experimentally measured adsorption amounts. To evaluate the performance of isotherm fitting, Root Mean Square Error (RMSE) is used, and it is expressed as:

$$RMSE = \sqrt{\frac{{\sum }_{i} {( {n}_{i}^{calc} - {n}_{i}^{expt} )}^{2}}{N}} \qquad ( 54 )$$

where N represents the total number of data points.

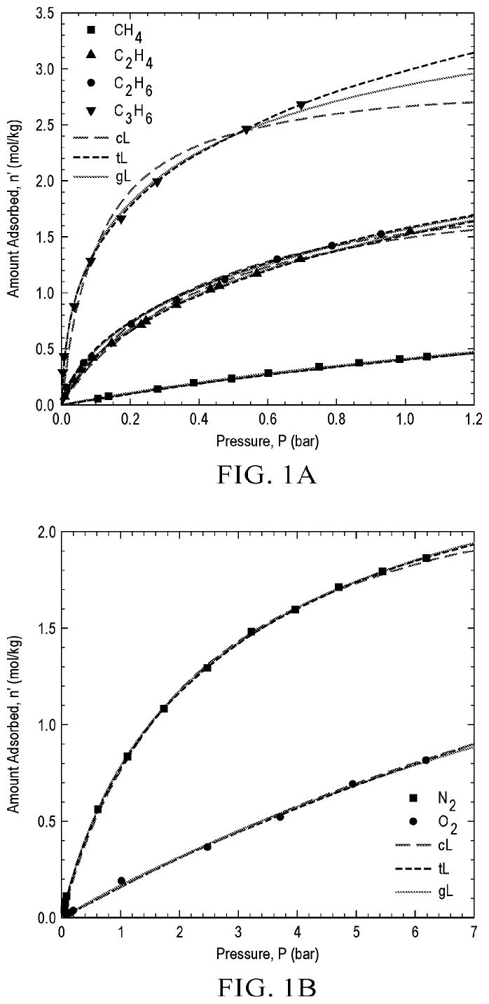

Nine pure component adsorption isotherm data sets are selected to support subsequent mixed-gas adsorption equilibria studies and they have been used to identify the model parameters of cL, tL, and gL pure component isotherms. These data include adsorption isotherms of CH₄, C₂H₄, C₂H₆, and C₃H₆ on activated carbon at 323 K, [28] N₂ and O₂ on LiLSX at 303.15 K [29], and CO₂, H₂S, and C₃H₈ on zeolite H-mordenite at 303.15 K [30]. The graphical representation of these data sets and the model results are shown in the FIGS. 1A to 1C. The corresponding regression results are summarized in Table 1, Table 2, and Table 3 for cL, tL, and gL, respectively. Note that the identification of tL isotherm parameters for systems involving only data in the Henry's law region, i.e., at low relative pressures, is more challenging due to lack of information on saturation capacities. For example, the much higher uncertainty in estimating nᵢ⁰ parameter of tL for O₂ on LiLSX, as shown in Table 2, can be attributed to the fact that the experimental data are limited to the Henry's law region.

TABLE 1
Regressed parameters for classical Langmuir isotherm

| | | T | K | nᵢ⁰ | RMSE | |
|---|---|---|---|---|---|---|
| Adsorbent | Adsorbate | (K) | (bar⁻¹) | (mol/kg) | (mol/kg) | Ref. |
| Activated | CH₄ | 323 | 0.402 ± 0.265 | 1.44 ± 0.70 | 0.003 | [28] |
| Carbon | C₂H₄ | 323 | 2.265 ± 0.196 | 2.14 ± 0.09 | 0.041 | |
| | C₂H₆ | 323 | 2.511 ± 0.271 | 2.13 ± 0.10 | 0.046 | |
| | C₃H₆ | 323 | 9.020 ± 0.366 | 2.96 ± 0.04 | 0.143 | |
| LiLSX | N₂ | 303.15 | 0.435 ± 0.029 | 2.54 ± 0.07 | 0.013 | [29] |
| | O₂ | 303.15 | 0.046 ± 0.029 | 3.70 ± 0.07 | 0.011 | |
| Zeolite H- | CO₂ | 303.15 | 7.459 ± 0.054 | 2.41 ± 0.01 | 0.129 | [30] |
| mordenite | H₂S | 303.15 | 102.193 ± 3.584 | 2.14 ± 0.02 | 0.120 | |
| | C₃H₈ | 303.15 | 33.413 ± 0.754 | 1.12 ± 0.01 | 0.107 | |

TABLE 2
Regressed parameters for thermodynamic Langmuir isotherm

| | | T | Kᵢ^{o} | nᵢ^{o} | | RMSE | |
|---|---|---|---|---|---|---|---|
| Adsorbent | Adsorbate | (K) | (bar⁻¹) | (mol/kg) | τ_{iϕ} | (mol/kg) | Ref. |
| Activated | CH₄ | 323 | 0.402 ± | 1.44 ± | −0.02 ± | 0.003 | [28] |
| Carbon | | | 0.292 | 0.78 | 8.06 | | |
| | C₂H₄ | 323 | 0.148 ± | 6.73 ± | −2.18 ± | 0.014 | |
| | | | 0.398 | 6.63 | 0.79 | | |
| | C₂H₆ | 323 | 0.193 ± | 6.13 ± | −2.21 ± | 0.021 | |
| | | | 0.480 | 5.55 | 0.71 | | |
| | C₃H₆ | 323 | 0.942 ± | 6.67 ± | −2.52 ± | 0.028 | |
| | | | 0.732 | 1.95 | 0.39 | | |
| LiLSX | N₂ | 303.15 | 0.344 ± | 2.83 ± | −0.91 ± | 0.006 | [29] |
| | | | 0.047 | 0.17 | 0.16 | | |
| | O₂ | 303.15 | 0.0033 ± | 15.01 ± | −1.77 ± | 0.011 | |
| | | | 0.045 | 85.14 | 4.56 | | |
| Zeolite H- | CO₂ | 303.15 | 1.632 ± | 4.14 ± | −2.42 ± | 0.016 | [30] |
| Mordenite | | | 0.544 | 0.44 | 0.11 | | |
| | H₂S | 303.15 | 75.082 ± | 2.78 ± | −3.09 ± | 0.022 | |
| | | | 10.181 | 0.11 | 0.19 | | |
| | C₃H₈ | 303.15 | 41.038 ± | 1.27 ± | −3.02 ± | 0.050 | |
| | | | 4.537 | 0.05 | 0.11 | | |

TABLE 3
Regressed parameters for generalized Langmuir isotherm

| | | T | Kᵢ^{o} | nᵢ^{o} | | RMSE | Aᵢ | |
|---|---|---|---|---|---|---|---|---|
| Adsorbent | Adsorbate | (K) | (bar⁻¹) | (mol/kg) | τ_{iϕ} | (mol/kg) | (nm²) | Ref. |
| Activated | CH₄ | 323 | 0.017 ± | 7.27 ** | −1.99 ± | 0.003 | 0.160 ³³ | [28] |
| Carbon | | | 0.015 | | 0.56 | | | |
| | C₂H₄ | 323 | 0.192 ± | 5.17 ** | −2.14 ± | 0.014 | 0.225 ³³ | |
| | | | 0.094 | | 0.17 | | | |
| | C₂H₆ | 323 | 0.196 ± | 5.17 ** | −1.85 ± | 0.021 | 0.225 ³³ | |
| | | | 0.104 | | 0.17 | | | |
| | C₃H₆ | 323 | 2.121 ± | 3.88 ** | −1.59 ± | 0.046 | 0.300 ³² | |
| | | | 1.793 | | 0.34 | | | |
| LiLSX | N₂ | 303.15 | 0.344 ± | 2.83 ± | −0.91 ± | 0.006 | 0.162 ²⁶ | [29] |
| | | | 0.047 | 0.17 | 0.16 | | | |
| | O₂ | 303.15 | 0.062 ± | 3.25 ** | −0.02 ± | 0.012 | 0.141 ²⁶ | |
| | | | 0.024 | | 1.59 | | | |
| Zeolite H- | CO₂ | 303.15 | 1.619 ± | 4.14 ± | −2.42 ± | 0.016 | 0.163 ²⁶ | [30] |
| Mordenite | | | 0.544 | 0.44 | 0.11 | | | |
| | H₂S | 303.15 | 26.553 ± | 3.21 ** | −3.10 ± | 0.028 | 0.210 ³⁵ | |
| | | | 27.009 | | 0.51 | | | |
| | C₂H | 303.15 | 0.877 ± | 2.25 ** | −3.74 + | 0.113 | 0.300 ³² | |
| | | | 2.477 | | 1.97 | | | |

** nᵢ^{o} is calculated from A^{o}

The results show both tL and gL perform much better than cL. In addition, while tL and gL show very similar RMSE's in fitting the data, some of the values of tL isotherm parameter nᵢ⁰ seem unreasonable. For example, the regressed tL nᵢ⁰ value for CH₄ adsorbed on activated carbon is exceedingly small and only a fraction (˜20%) of that of C₂H₄. In contrast, given a constant A^{o} for an adsorbent, the values of gL isotherm parameter nᵢ⁰ are inversely proportional to the values of Aᵢ. Note that the adsorbate saturation loadings, i.e., nᵢ⁰'s, on activated carbon shown in Table 3 are calculated from the reported adsorbent surface area of

$$700 \frac{{m}^{2}}{g} [ 28 , 31 ]$$

and the corresponding adsorbate effective areas in the literature [32, 33]. For LiLSX, Aᵢ is known for N₂ [26], nᵢ⁰ for N₂ is fitted to the isotherm data, and A^{o} is then calculated to be

$$276 \frac{{m}^{2}}{g} .$$

For zeolite H-mordenite, Aᵢ is known for CO₂ [26], nᵢ⁰ for CO₂ is fitted to the isotherm data, and A^{o} is then calculated to be

$$406 \frac{{m}^{2}}{g} ,$$

consistent with the literature value of

$$429 \frac{{m}^{2}}{g} . \qquad [ 34 ]$$

### Binary Mixed-Gas Adsorption Equilibria

Ten binary mixed-gas adsorption systems have been investigated with eL, IAST, and gL. The regressed gL parameters and the corresponding RMSE's for all the binary mixed-gas adsorption systems with the three models are reported in Table 4. For internal model consistency, the cL model is used to calculate pure component adsorption isotherms in the eL calculations for mixed-gas adsorption equilibria. The tL model is used to calculate pure component adsorption isotherms and the corresponding spreading pressures in the IAST calculations. In contrast, the gL isotherm is used in both pure component adsorption isotherms and mixed-gas adsorption equilibria.

TABLE 4
Model results for binary mixed-gas adsorption equilibria

| | | T | P | IAST | eL | gL | | |
|---|---|---|---|---|---|---|---|---|
| Adsorbent | Adsorbates | (K) | (bar) | RMSE | RMSE | τ₁₂ | RMSE | Ref. |
| Activated | CH₄(1)—C₂H₄(2) | 323 | 0.1 | 0.059 | 0.114 | 0 | 0.062 | [28] |
| Carbon | CH₄(1)—C₂H₆(2) | 323 | 0.1 | 0.161 | 0.233 | 0 | 0.163 | |
| | C₂H₄(1)—C₂H₆(2) | 323 | 0.1 | 0.041 | 0.061 | −0.54 ± | 0.044 | |
| | | | | | | 0.00 | | |
| | C₂H₄(1)—C₃H₆(2) | 323 | 0.1 | 0.080 | 0.196 | −1.34 ± | 0.084 | |
| | | | | | | 0.00 | | |
| | C₂H₆(1)—C₃H₆(2) | 323 | 0.1 | 0.040 | 0.128 | −2.36 ± | 0.036 | |
| | | | | | | 0.01 | | |
| LiLSX | N₂(1)—O₂(2) | 303.15 | 1.013 | 0.013 | 0.004 | −1.83 ± | 0.005 | [29] |
| | | | | | | 0.00 | | |
| | N₂(1)—O₂(2) | 303.15 | 6.080 | 0.015 | 0.028 | −1.83 ± | 0.001 | |
| | | | | | | 0.00 | | |
| Zeolite H- | H₂S (1)—CO₂(2) | 303.15 | 0.156 | 0.049 | 0.044 | −2.95 ± | 0.013 | [30] |
| mordenite | | | | | | 0.00 | | |
| | C₃H₈ (1)—H₂S(2) | 303.15 | 0.081 | 0.145 | 0.181 | −3.93 ± | 0.038 | |
| | | | | | | 0.00 | | |
| | C₃H₈ (1)—CO₂(2) | 303.15 | 0.410 | 0.162 | 0.212 | −4.87 ± | 0.085 | |
| | | | | | | 0.00 | | |

Five binary mixed-gas adsorption of hydrocarbons on activated carbon at 323 K and 0.1 bar [28] are investigated. gL estimations accurately match the experimental data and outperform eL predictions for the five binary systems of CH₄—C₂H₄, CH₄—C₂H₆, C₂H₄—C₂H₆, C₂H₄—C₃H₆, and C₂H₆—C₃H₆. RMSEs of IAST and gL are same for all five binary systems on activated carbon.

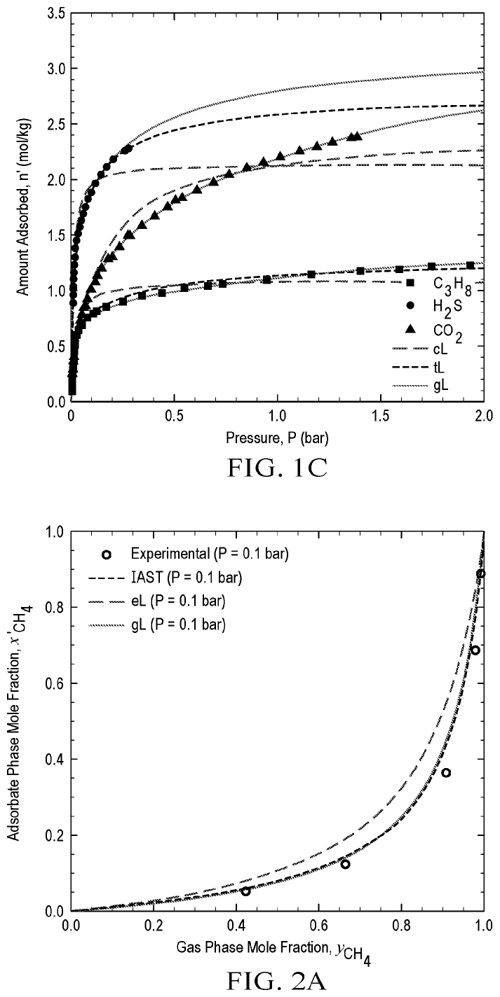

FIGS. 2A to 2E show the experimental data [28] and the model results for the five binary systems on activated carbon at 323 K and 0.1 bar [28]: (FIG. 2A) CH₄—C₂H₄, (FIG. 2B) CH₄—C₂H₆, (FIG. 2C) C₂H₄—C₂H₆, (FIG. 2D) C₂H₄—C₃H₆, and (FIG. 2E) C₂H₆—C₃H₆. The eL predictions deviate much from the experimental adsorbate phase composition while the IAST and gL results match or are close to the data. Note that the adsorbate phase mole fractions, x, calculated from gL with Eqs. (18)-(19) are referred as “true” adsorbate phase mole fractions and they can be transformed to “apparent” adsorbate phase mole fractions, xᵢ, upon renormalization with x_{ϕ} excluded. “Apparent” adsorbate phase mole fractions are used to generate all the figures herein.

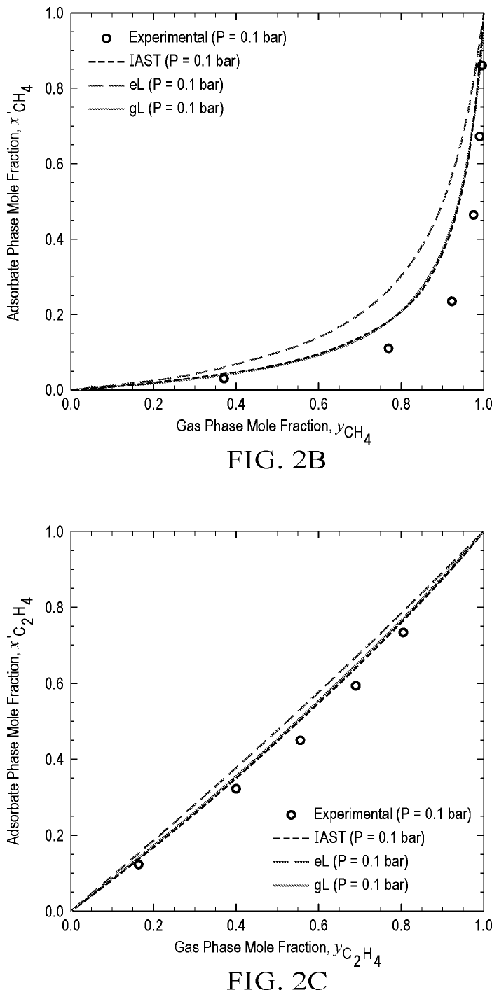

FIG. 3 presents the experimental data [29] and the model results for the mixed-gas adsorption of N₂—O₂ binary mixture on LiLSX at pressure of 1.013 bar and 6.08 bar at 303.15 K.²⁹ While eL satisfactorily predicts the mixed-gas adsorption equilibria at 1.013 bar, eL does not address the system pressure effect and its predictions for 6.08 bar remain unchanged from those for 1.013 bar and significantly depart from the experimental data. Although the IAST predictions show correct pressure dependence, the IAST predictions for both 1.013 bar and 6.08 bar deviate from the experimental data. Interestingly, gL successfully correlates the adsorption equilibria data at 6.08 bar with τ₁₂=−1.83, and then accurately predicts the adsorption equilibria at 1.013 bar.

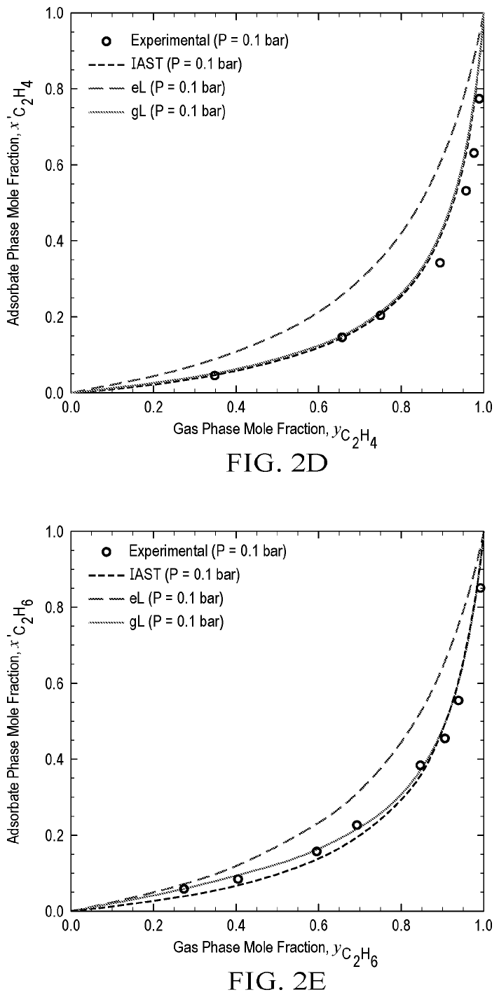

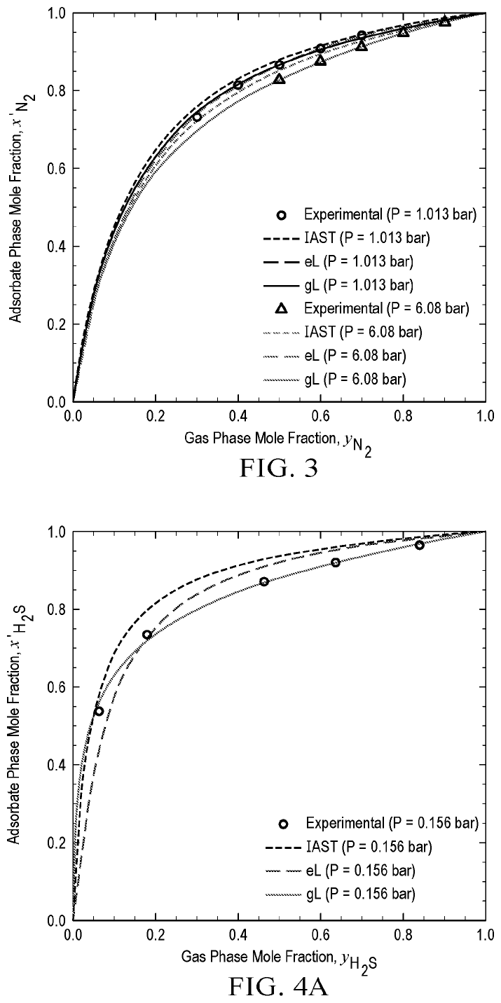

FIG. 4A shows the experimental data [30] and the model results for mixed-gas adsorption of H₂S—CO₂ binary mixture on zeolite H-mordenite at 303.15 K and 0.156 bar. Neither IAST nor eL predict the adsorption behavior while gL accurately correlates the mixed-gas adsorption equilibria with τ₁₂=−2.95. FIG. 4B and FIG. 4C show the adsorption azeotropic behaviors of C₃H₈—H₂S and C₃H₈—CO₂ binary gas mixtures at 303.15 K on zeolite H-mordenite at 0.081 bar and 0.41 bar, respectively. For these azeotropes-forming systems, both IAST and eL fail to predict the azeotropic behaviors while gL reliably captures the adsorption azeotropes with τ₁₂=−3.93 for the C₃H₈—H₂S binary and τ₁₂=−4.87 for the C₃H₈—CO₂ binary.

Estimate Surface Loading from Adsorption Equilibria

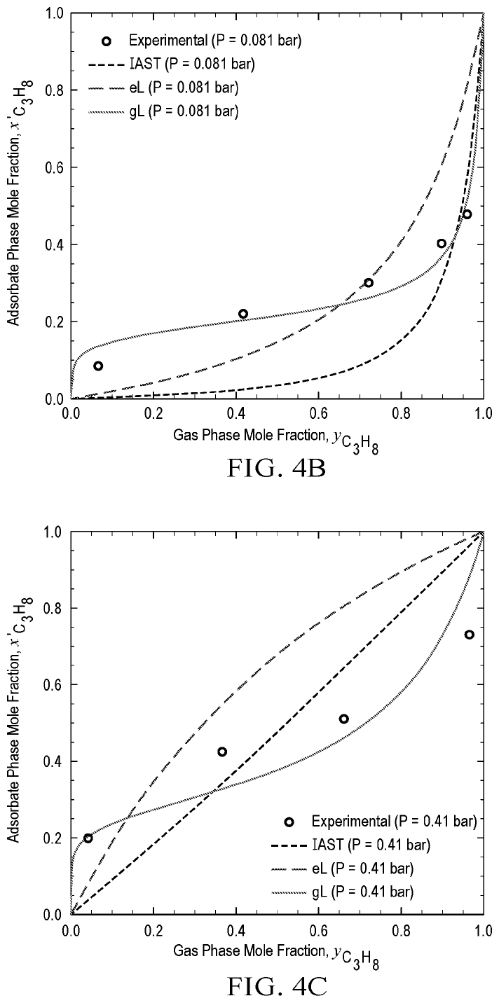

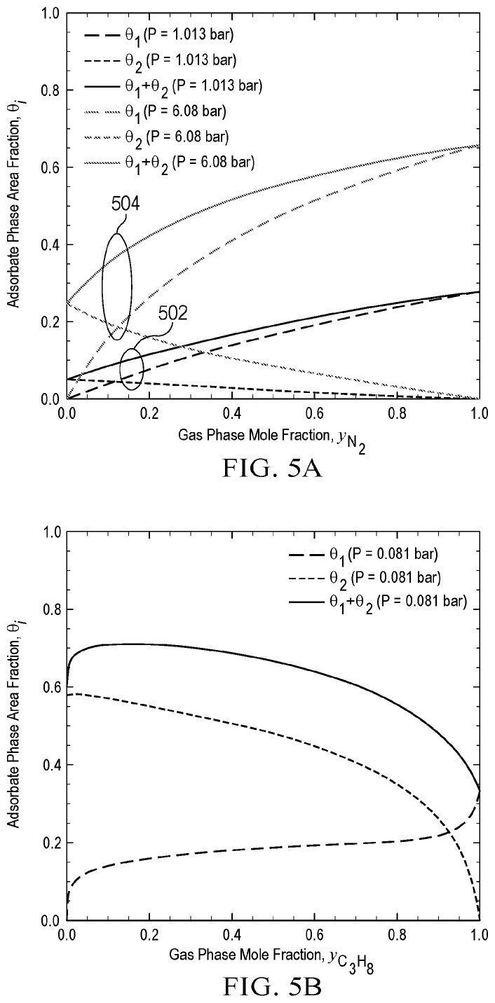

The unique feature of gL to consider adsorbent as a part of adsorption system makes the tracking of adsorbent surface loading possible. For the adsorption of N₂—O₂ binary on LiLSX at 1.013 bar and 6.08 bar and at 303.15 K [29], FIG. 5A shows the overall surface loading jumps when the system pressure changes from 1.013 bar (black lines denoted as 502) to 6.08 bar (red lines denoted as 504). It further shows that at low pressure, i.e., 1.013 bar, N₂ adsorption and O₂ adsorption are relatively independent of each other. As the gas phase N₂ mole fraction increases, the N₂ adsorbate phase area fraction increases linearly, albeit with a slight positive deviation, while the O₂ adsorbate phase area fraction declines linearly. At high pressure, i.e., 6.08 bar, the adsorption competition between O₂ and N₂ strengthens. The slight positive deviation from the linear relationship for the N₂ adsorbate phase area fraction vs the gas phase N₂ mole fraction is magnified while the O₂ adsorbate phase area fraction shows negative deviation from the linear relationship.

For the two binary mixtures exhibiting azeotropic behavior, i.e., C₃H₈—H₂S binary on zeolite H-mordenite at 0.081 bar and 303.15 K [30] and C₃H₈—CO₂ binary on zeolite H-mordenite at 0.41 bar and 303.15 K [30], FIG. 5B and FIG. 5C show both H₂S and CO₂ adsorptions drop as their gas phase mole fractions drop while, responsible for the azeotropic behavior, the C₃H₈ adsorbate phase area fractions show logit S-shape behavior as the gas phase C₃H₈ mole fractions increase. Interestingly, both these two azeotrope-forming systems exhibit a maximum total absorbent surface coverage at the azeotropes.

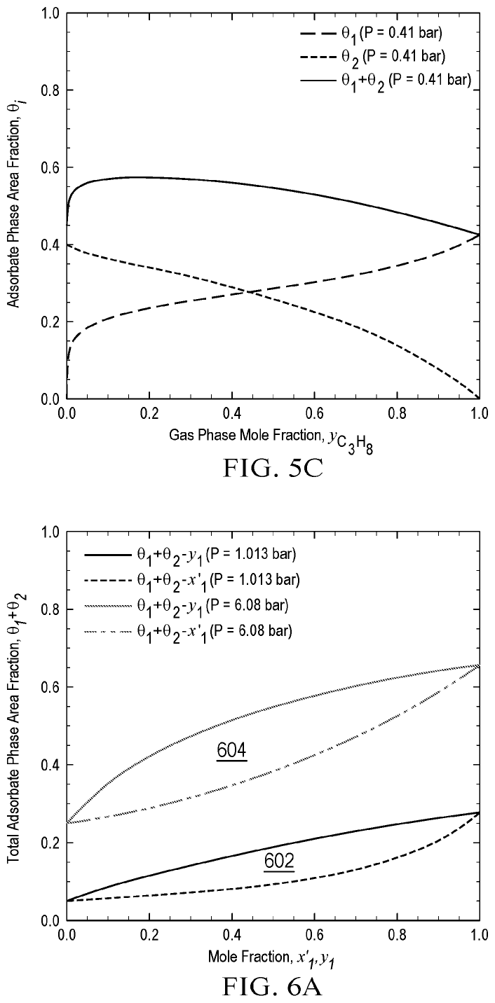

The mixed-gas adsorption equilibria phase diagram analogous to P-xy phase diagram in vapor-liquid equilibria can be generated from gL. FIG. 6A shows the θ-x′y phase diagrams of N₂—O₂ binary at 1.013 bar (lower pair of black lines denoted as 602) and 6.08 bar (upper pair of red lines denoted as 604) on LiLSX at 303.15 K. Here θ is the overall surface loading, i.e., (θ₁+θ₂). The θ-x′y phase diagram of N₂—O₂ binary at 1.013 bar shows nearly “Raoult's law” type phase diagram and the nonideality is much enhanced for the system at 6.08 bar. FIG. 6B shows the θ-x′y phase diagrams of C₃H₈—H₂S binary at 0.081 bar (upper pair of black lines denoted as 606) and C₃H₈—CO₂ binary at 0.41 bar (lower pair of red lines denoted as 608) on zeolite H-mordenite at 303.15 K. At azeotropes, the systems exhibit maximum overall surface loading and the apparent adsorbate phase component mole fractions equal to the gas phase component mole fractions.

Estimate Activity Coefficients from Adsorption Equilibria

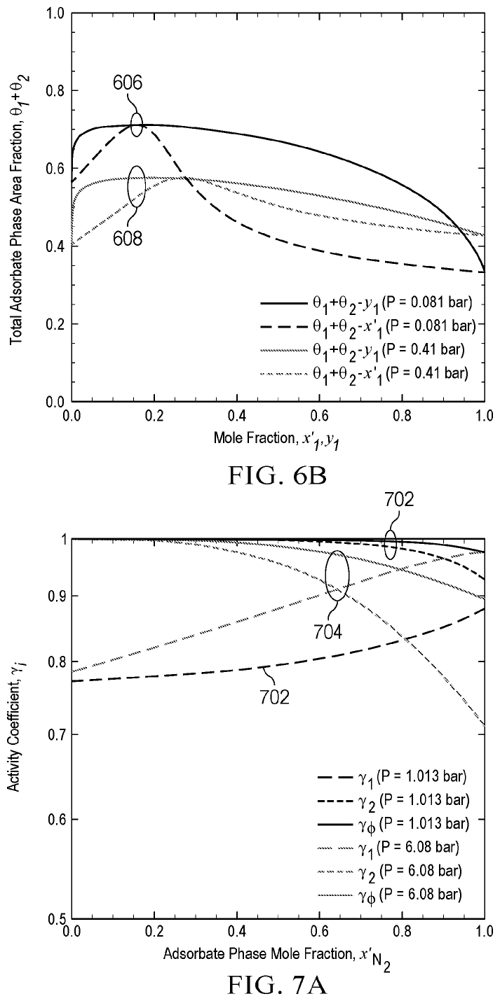

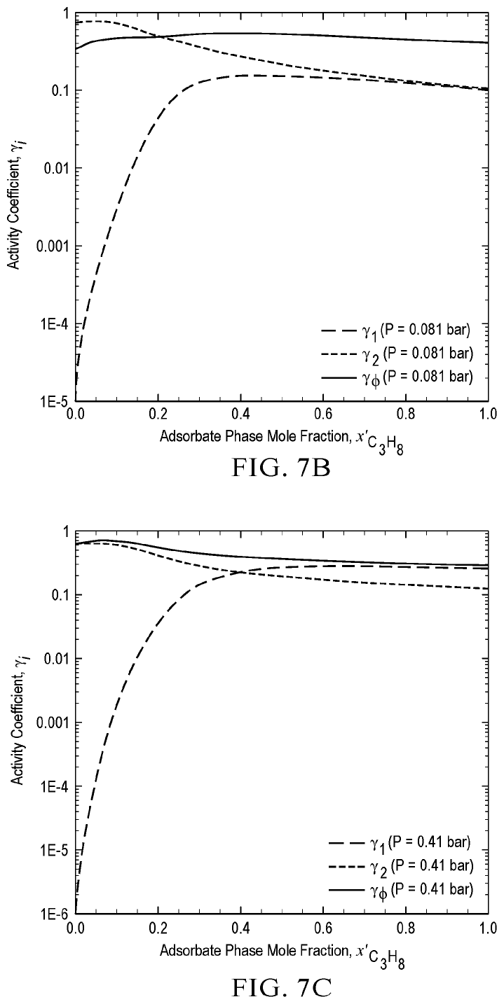

Besides tracking the adsorbent surface loading, gL captures the adsorbent surface heterogeneity by tracking the adsorbate phase activity coefficients of adsorbates and vacant sites. As previously mentioned, full adsorbent surface coverage of pure adsorbate molecules is defined as the reference state for adsorbates. For the adsorption of N₂—O₂ binary on LiLSX at 1.013 (black lines denoted as 702) and 6.08 bar (red lines denoted as 704), and 303.15 K [29], FIG. 7A shows the activity coefficients of adsorbates and vacant sites as function of apparent adsorbate phase N₂ mole fraction. With τ_{1ϕ}≈0 for O₂, activity coefficients for O₂ and vacant sites remain close to unity until the adsorbent surface is significantly covered with N₂. On the other hand, activity coefficient for N₂ increases and approaches unity as the N₂ adsorbate phase area fraction increases with increasing apparent adsorbate phase mole fraction and system pressure.

FIG. 7B and FIG. 7C show the activity coefficients of adsorbates and vacant sites as function of apparent adsorbate phase C₃H₈ mole fraction for the C₃H₈—H₂S binary on zeolite H-mordenite at 0.081 bar and 303.15 K [30] and the C₃H₈—CO₂ binary at 0.41 bar and 303.15 K [30]. With t=−3.74 for C₃H₈, the activity coefficient of C₃H₈ at infinite dilution is very small for both binary systems, indicative of strong attractive interaction between the adsorbate and the adsorbent. The activity coefficient of C₃H₈ then increases sharply and reaches a plateau as its apparent adsorbate phase mole fraction increases. As expected, the corresponding activity coefficients of CO₂ and H₂S in their respective binary adsorption systems with C₃H₈ are close to unity (˜0.8) when their apparent adsorbate phase mole fractions are unity. The activity coefficients then drop to around 0.1 as their respective apparent adsorbate phase mole fraction approaches zero. The activity coefficients of vacant sites remain relatively unchanged since the overall surface loadings, θ's, stay relatively constant. The vacant sites activity coefficients hover around 0.4 to 0.5 for the C₃H₈—H₂S binary and drop from 0.7 to 0.3 for the C₃H₈—CO₂ binary as the C₃H₈ apparent adsorbate phase mole fractions vary from zero to unity.

### Prediction of Mixed-Gas Adsorption Equilibria

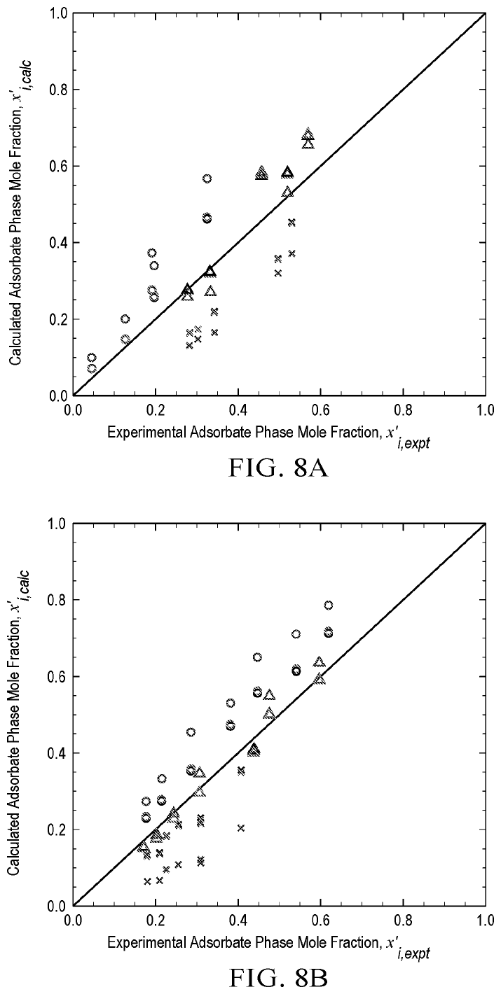

To examine the predictive capability of gL, the mixed-gas adsorption equilibria is predicted for CH₄—C₂H₄—C₂H₆ ternary and C₂H₄—C₂H₆—C₃H₆ ternary on activated carbon at 323 K and 0.1 bar [28] and CO₂— H₂S—C₃H₈ ternary on zeolite H-mordenite at 303.15 K and 0.007-0.1342 bar [30]. The predictions with eL, IAST, and gL are based on the pure component adsorption isotherm parameters reported in Table 1, Table 2, and Table 3 and the regressed τᵢⱼ's reported in Table 4. The parity plots of above mentioned mixed-gas adsorption equilibria predictions for apparent adsorbate phase compositions are presented in FIGS. 8A-8C. FIG. 8A depicts CH₄ (∘)-C₂H₄(Δ)-C₂H, (x) on activated carbon at 323 K and 0.1 bar [28]. FIG. 8B depicts C₂H₄(∘)-C₂H, (Δ)-C₃H₆(x) on activated carbon at 323 K and 0.1 bar [28]. FIG. 8C depicts CO₂ (∘)-H₂S (Δ)-C₃H₈ (x) on zeolite H-mordenite at 303.15 K and 0.007-0.1342 bar [30]. The blue, black, and red colors represent eL, IAST, and gL predictions, respectively.

The average relative deviation (ARD %) results are reported in Table 5.

$$ARD \% = \frac{100}{N} {\sum }_{i = 1}^{N} \vert \frac{{x}_{i , calc}^{′} - {x}_{i , expt}^{′}}{{x}_{i , expt}^{′}} \vert \qquad ( 55 )$$

TABLE 5
Average relative deviation (ARD %) of ternary mixed-gas
adsorption equilibria systems

| Adsorbents | Adsorbates | IAST | eL | gL |
|---|---|---|---|---|
| Activated Carbon at 323K and | CH₄(1)—C₂H₄(2)—C₂H₆(3) | 27.2% | 47.1% | 27.5% |
| 0.1 bar ²⁸ | C₂H₄(1)—C₂H₆(2)—C₃H₆(3) | 17.6% | 37.7% | 18.6% |
| Zeolite H-mordenite at 303.15K | CO₂(1)—H₂S(2)—C₃H₈(3) | 48.8% | 27.0% | 19.3% |
| and 0.007-0.1342 bar ³⁰ | | | | |

For mixed-gas adsorption equilibria for CH₄—C₂H₄—C₂H₆ ternary and C₂H₄—C₂H₆-C₃H₆ ternary on activated carbon at 323 K and 0.1 bar [28], the eL predictions have the highest ARD's of 47% and 38%, respectively. IAST and gL provide very similar predictions for CH₄—C₂H₄-C₂H₆ ternary and C₂H₄—C₂H₆—C₃H₆ ternary on activated carbon with ARD around 27% and 18%, respectively. In case of mixed-gas adsorption for CO₂— H₂S—C₃H₈ ternary on zeolite H-mordenite at 303.15 K and 0.007-0.1342 bar [30], the IAST predictions yield the highest ARD of nearly 49%, followed by the eL predictions with ARD of 27%. The gL predictions yield the best predictions with ARD of 19%.

Comparison of IAST-aNRTL and generalized Langmuir capabilities

In addition to the comparison with eL and IAST, the gL results have also been checked against our prior investigation for mixed-gas adsorption equilibria based a modified IAST and the original adsorption NRTL model [10]. Specifically, Table 6 reports the RMSEs for IAST-aNRTL and gL for five binary systems: N₂—O₂ binary at 1.013 bar and at 6.08 bar on LiLSX at 303.15 K [29], H₂S—CO₂ binary at 0.156 bar, C₃H₈—H₂S binary at 0.081 bar, and C₃H₈—CO₂ binary at 0.41 bar on zeolite H-mordenite at 303.15 K. [30] Overall, the gL results for the mixed-gas adsorption equilibria are slightly better than the IAST-aNRTL results. More importantly, the gL results are thermodynamically consistent while the IAST-aNRTL results are valid at constant spreading pressures even though the adsorption equilibria are at constant system pressures. Moreover, gL does not require the computationally expansive calculations on spreading pressures as required for IAST-aNRTL.

TABLE 6
Regressed IAST-aNRTL and generalized Langmuir parameters
for binary mixed-gas adsorption equilibria

| | | T | P | IAST-aNRTL ¹⁰ | | gL | | |
|---|---|---|---|---|---|---|---|---|
| Adsorbent | Adsorbates | (K) | (bar) | τ₁₂ | RMSE | τ₁₂ | RMSE | Ref. |
| LiLSX | N₂(1)—O₂(2) | 303.15 | 1.013 | −0.84 ± | 0.006 | −1.83 ± | 0.005 | [29] |
| | | | | 0.04 | | 0.00 | | |
| | N₂(1)—O₂(2) | 303.15 | 6.080 | −0.84 ± | 0.005 | −1.83 ± | 0.001 | |
| | | | | 0.04 | | 0.00 | | |
| Zeolite H- | H₂S (1)—CO₂(2) | 303.15 | 0.156 | −1.60 ± | 0.020 | −2.95 ± | 0.013 | [30] |
| Mordenite | | | | 0.31 | | 0.00 | | |
| | C₃H₈(1)—H₂S(2) | 303.15 | 0.081 | −3.22 ± | 0.042 | −3.93 ± | 0.038 | |
| | | | | 0.02 | | 0.00 | | |
| | C₃H₈ (1)—CO₂(2) | 303.15 | 0.410 | −3.77 ± | 0.063 | −4.87 ± | 0.085 | |
| | | | | 0.02 | | 0.00 | | |

### CONCLUSION

A simple, robust, and thermodynamically consistent adsorption model for pure component adsorption isotherms and mixed-gas adsorption equilibria has been described herein. Treating the adsorbent surface as an integral part of adsorption systems, the proposed generalized Langmuir isotherm accurately correlates and predicts mixed-gas adsorption equilibria without the need to compute “spreading pressure” as required for Ideal Adsorbed Solution Theory. Tested with mixed-gas adsorption equilibria data for ten binary and three ternary systems, the generalized Langmuir isotherm outperforms both extended Langmuir and Ideal Adsorbed Solution Theory in predicting mixed-gas adsorption equilibria. As the generalized Langmuir isotherm tracks the surface loading in terms of occupied and vacant sites surface area fractions and the surface heterogeneity in terms of the adsorbate phase activity coefficients, it is shown for the first-time complete phase diagrams for mixed-gas adsorption equilibria analogous to the TPxy phase diagrams for vapor-liquid equilibria. Additionally, the generalized Langmuir isotherm predictions for mixed-gas adsorption equilibria can be used to elucidate the spreading pressure dependence of adsorbate phase activity coefficients in the context of Ideal Adsorbed Solution Theory.

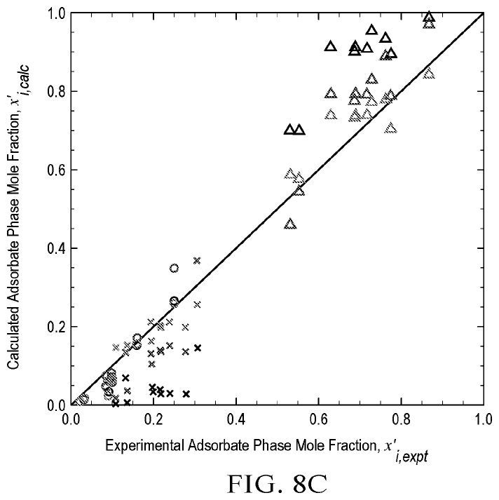

Some other embodiments of the present invention will now be described with respect to FIGS. 9-11. FIG. 9 is a block diagram of an apparatus, system or computer 900, such as a workstation, laptop, desktop, tablet computer, mainframe, or other single or distributed computing platform suitable for performing the methods described herein. Note that the components can be integrated into a single device or communicably coupled to one another via a network. The apparatus, system or computer 900 includes one or more processors 902, a memory or data storage 904, and one or more communication interfaces or input/output interfaces 906, which can be communicably coupled to one or more output device(s) 908 (e.g., printer, internal or external data storage device, display or monitor, remote database, remote computer, etc.) via a network or communications link 910 (e.g., wired, wireless, optical, etc.). The one or more output device(s) can be integrated into the computer 900 as indicated by the dashed line 912

The apparatus, system or computer 900 can be used to estimate an adsorption equilibria for one or more gases from pure component adsorption isotherms. The one or more processors calculate an adsorption of each gas i on a constant monolayer adsorption surface A^{o} using generalized Langmuir isotherm equations:

$${θ}_{i} = \frac{{n}_{i} {A}_{i}}{{A}^{o}} = \frac{{K}_{i}^{o} {y}_{i} P}{\frac{{γ}_{i}}{{γ}_{ϕ} {q}_{i}} + {\sum }_{j = 1}^{n} \frac{{γ}_{i} {q}_{j}}{{γ}_{j} {q}_{i}} {K}_{j}^{o} {y}_{j} P}$$

$${θ}_{ϕ} = \frac{{n}_{ϕ} {A}_{ϕ}}{{A}^{o}} = \frac{1}{1 + {\sum }_{j = 1}^{n} \frac{{γ}_{ϕ} {q}_{j}}{{γ}_{j}} {K}_{j}^{o} {y}_{j} P}$$

$${q}_{i} = \frac{{A}_{i}}{{A}_{ϕ}}$$

where: θᵢ is an adsorbate phase area fraction covered with the gas i,
nᵢAᵢ is an occupied area for the gas i,
Kᵢ^{o} is an intrinsic adsorption equilibrium constant of the gas i,
yᵢ is a gas phase mole fraction of gas i,
P is a gas vapor pressure,
γᵢ is an activity coefficient of the gas i,
γ_{ϕ} is an activity coefficient of vacant sites,
qᵢ is a ratio of an effective area of the gas i (Aᵢ) and an effective area of a phantom molecule ϕ (A_{ϕ}),
n is a number of the one or more gases,
θ_{ϕ} is an adsorbate phase vacant site area fraction, and
n_{ϕ}A_{ϕ} is a vacant area for the phantom molecule ϕ.

The adsorption of each gas i is provided to the output device 908, and a chemical process or a product is developed using the adsorption of each gas i.

In one aspect, the generalized Langmuir isotherm equations reduce to

$$\frac{{n}_{i}}{{n}_{i}^{0}} = \frac{{K}_{i}^{o} {y}_{i} P}{1 + {\sum }_{i = 1}^{n} {K}_{i}^{o} {y}_{i} P}$$

when (1) the adsorbate and vacant site effective areas are the same A₁=A₂= . . . =Aᵢ=A_{ϕ}, or equivalently, the saturation loadings of adsorbates and phantom molecule are same n₁⁰=n₂⁰= . . . =nᵢ⁰=n_{ϕ}⁰, and (2) the adsorbate phase activity coefficients are unity γᵢ=γ_{ϕ}=1. In another aspect, the one or more gases comprise a mixed gas having two or more components. In another aspect, the constant monolayer adsorption surface comprises activated carbon, LiLSX or Zeolite H-mordenite. In another aspect, the gas i comprises CH₄, C₂H₄, C₂H₆, C₃H₆, N₂, O₂, CO₂, H₂S, or C₃H₈. In another aspect, the one or more gasses comprise a mixed gas selected from CH₄—C₂H₄, CH₄—C₂H₆, C₂H₄—C₂H₆, C₂H₄—C₂H₆, and C₂H₆—C₃H₆. In another aspect, the one or more gasses comprise a mixed gas selected from N₂ and O₂. In another aspect, the one or more gasses comprise a mixed gas selected from H₂S—CO₂, C₃H₈—H₂S, and C₃H₈—CO₂.

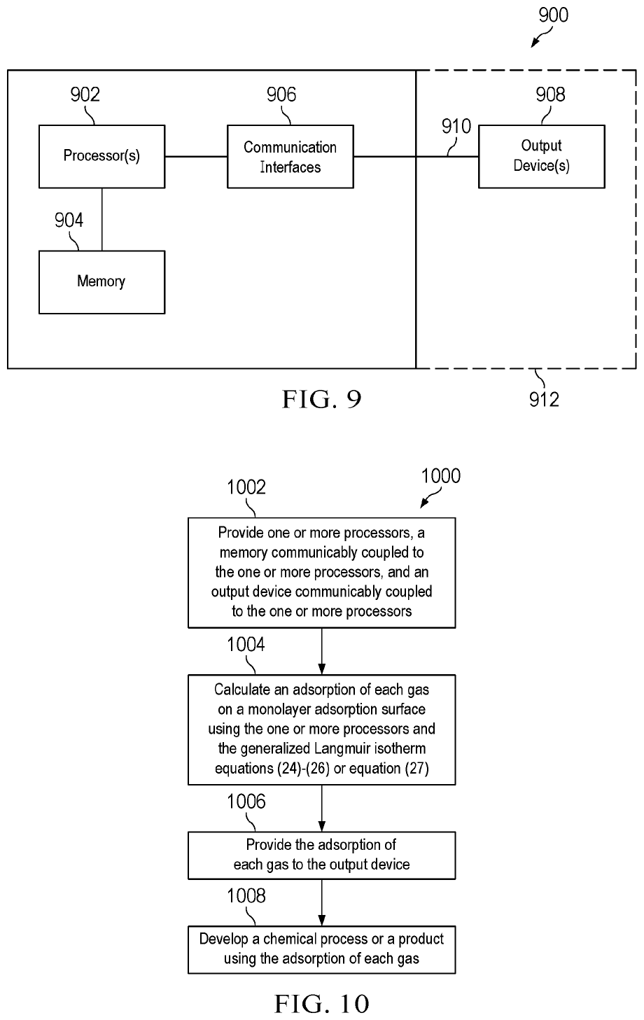

FIG. 10 is a flow chart depicting a computerized method 1000 for estimating an adsorption equilibria for one or more gases from pure component adsorption isotherms. One or more processors, a memory communicably coupled to the one or more processors and an output device communicably coupled to the one or more processors are provided in block 1002. An adsorption of each gas on a constant monolayer adsorption surface is calculated using the one or more processors and the generalized Langmuir isotherm equations (24)-(26) or equation (27) in block 1004, namely:

$${θ}_{i} = \frac{{n}_{i} {A}_{i}}{{A}^{o}} = \frac{{K}_{i}^{o} {y}_{i} P}{\frac{{γ}_{i}}{{γ}_{ϕ} {q}_{i}} + {\sum }_{j = 1}^{n} \frac{{γ}_{i} {q}_{j}}{{γ}_{j} {q}_{i}} {K}_{j}^{o} {y}_{j} P}$$

$${θ}_{ϕ} = \frac{{n}_{ϕ} {A}_{ϕ}}{{A}^{o}} = \frac{1}{1 + {\sum }_{j = 1}^{n} \frac{{γ}_{ϕ} {q}_{j}}{{γ}_{j}} {K}_{j}^{o} {y}_{j} P}$$

$${q}_{i} = \frac{{A}_{i}}{{A}_{ϕ}}$$

where: θᵢ is an adsorbate phase area fraction covered with the gas i,
nᵢAᵢ is an occupied area for the gas i,
Kᵢ^{o} is an intrinsic adsorption equilibrium constant of the gas i,
yᵢ is a gas phase mole fraction of gas i,
P is a gas vapor pressure,
γᵢ is an activity coefficient of the gas i,
γ_{ϕ} is an activity coefficient of vacant sites,
qᵢ is a ratio of an effective area of the gas i (Aᵢ) and an effective area of a phantom molecule ϕ (A_{ϕ}),
n is a number of the one or more gases,
θ_{ϕ} is an adsorbate phase vacant site area fraction, and
n_{ϕ}A_{ϕ} is a vacant area for the phantom molecule ϕ.

The adsorption of each gas is provided to the output device in block 1006, and a chemical process or product is developed using the adsorption of each gas in block 1008. The method 1000 can be implemented by the apparatus 900 or by a non-transitory computer readable medium encoded with a computer program for execution by a processor that performs the steps of the method 1000.

In one aspect, the generalized Langmuir isotherm equations reduce to

$$\frac{{n}_{i}}{{n}_{i}^{0}} = \frac{{K}_{i}^{o} {y}_{i} P}{1 + {\sum }_{i = 1}^{n} {K}_{i}^{o} {y}_{i} P}$$

when (1) the adsorbate and vacant site effective areas are the same A₁=A₂= . . . =Aᵢ=A_{ϕ}, or equivalently, the saturation loadings of adsorbates and phantom molecule are same n₁⁰=n₂⁰= . . . =nᵢ⁰?=n_{ϕ}⁰, and (2) the adsorbate phase activity coefficients are unity γᵢ=γ_{ϕ}=1. In another aspect, the one or more gases comprise a mixed gas having two or more components. In another aspect, the constant monolayer adsorption surface comprises activated carbon, LiLSX or Zeolite H-mordenite. In another aspect, the gas i comprises CH₄, C₂H₄, C₂H₄, C₂H₆, N₂, O₂, CO₂, H₂S, or C₃H₈. In another aspect, the one or more gasses comprise a mixed gas selected from CH₄—C₂H₄, CH₄—C₂H₆, C₂H₄—C₂H₆, C₂H₄—C₃H₆, and C₂H₆—C₃H₆. In another aspect, the one or more gasses comprise a mixed gas selected from N₂ and O₂. In another aspect, the one or more gasses comprise a mixed gas selected from H₂S—CO₂, C₃H₈—H₂S, and C₃H₈—CO₂.

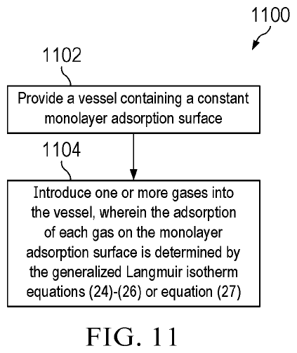

FIG. 11 is a flow chart depicting a method 1100 of adsorbing one or more gases. A vessel containing a constant monolayer adsorption surface is provided in block 1102. One or more gases are introduced into the vessel in block 1104, wherein the adsorption of each gas on the constant monolayer adsorption surface is determined by the generalized Langmuir isotherm equations (24)-(26) or equation (27), namely:

$${θ}_{i} = \frac{{n}_{i} {A}_{i}}{{A}^{o}} = \frac{{K}_{i}^{o} {y}_{i} P}{\frac{{γ}_{i}}{{γ}_{ϕ} {q}_{i}} + {\sum }_{j = 1}^{n} \frac{{γ}_{i} {q}_{j}}{{γ}_{j} {q}_{i}} {K}_{j}^{o} {y}_{j} P}$$

$${θ}_{ϕ} = \frac{{n}_{ϕ} {A}_{ϕ}}{{A}^{o}} = \frac{1}{1 + {\sum }_{j = 1}^{n} \frac{{γ}_{ϕ} {q}_{j}}{{γ}_{j}} {K}_{j}^{o} {y}_{j} P}$$

$${q}_{i} = \frac{{A}_{i}}{{A}_{ϕ}}$$

where: θᵢ is an adsorbate phase area fraction covered with the gas i,
nᵢAᵢ is an occupied area for the gas i,
Kᵢ^{o} is an intrinsic adsorption equilibrium constant of the gas i,
yᵢ is a gas phase mole fraction of gas i,
P is a gas vapor pressure,
γᵢ is an activity coefficient of the gas i,
γ_{ϕ} is an activity coefficient of vacant sites,
qᵢ is a ratio of an effective area of the gas i (Aᵢ) and an effective area of a phantom molecule ϕ (A_{ϕ}),
n is a number of the one or more gases,
θ_{ϕ} is an adsorbate phase vacant site area fraction, and
n_{ϕ}A_{ϕ} is a vacant area for the phantom molecule ϕ.
   A product can be produced in accordance with the method 1100.

In one aspect, the generalized Langmuir isotherm equations reduce to

$$\frac{{n}_{i}}{{n}_{i}^{0}} = \frac{{K}_{i}^{o} {y}_{i} P}{1 + {\sum }_{i = 1}^{n} {K}_{i}^{o} {y}_{i} P}$$

when (1) the adsorbate and vacant site effective areas are the same A₁=A₂= . . . =Aᵢ=A_{ϕ}, or equivalently, the saturation loadings of adsorbates and phantom molecule are same n₁⁰=n₂⁰= . . . =nᵢ⁰=n_{ϕ}⁰, and (2) the adsorbate phase activity coefficients are unity γᵢ=γ_{ϕ}=1. In another aspect, the one or more gases comprise a mixed gas having two or more components. In another aspect, the constant monolayer adsorption surface comprises activated carbon, LiLSX or Zeolite H-mordenite. In another aspect, the gas i comprises CH₄, C₂H₄, C₂H₆, C₃H₆, N₂, O₂, CO₂, H₂S, or C₃H₈. In another aspect, the one or more gasses comprise a mixed gas selected from CH₄—C₂H₄, CH₄—C₂H₆, C₂H₄—C₂H₆, C₂H₄—C₃H₆, and C₂H₆—C₃H₆. In another aspect, the one or more gasses comprise a mixed gas selected from N₂ and O₂. In another aspect, the one or more gasses comprise a mixed gas selected from H₂S—CO₂, C₃H₆—H₂S, and C₃H₈—CO₂.

It will be understood that particular embodiments described herein are shown by way of illustration and not as limitations of the invention. The principal features of this invention can be employed in various embodiments without departing from the scope of the invention. Those skilled in the art will recognize or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited features, elements, components, groups, integers, and/or steps, but do not exclude the presence of other unstated features, elements, components, groups, integers and/or steps. In embodiments of any of the compositions and methods provided herein, “comprising” may be replaced with “consisting essentially of” or “consisting of”. As used herein, the term “consisting” is used to indicate the presence of the recited integer (e.g., a feature, an element, a characteristic, a property, a method/process step or a limitation) or group of integers (e.g., feature(s), element(s), characteristic(s), property(ies), method/process steps or limitation(s)) only. As used herein, the phrase “consisting essentially of” requires the specified features, elements, components, groups, integers, and/or steps, but do not exclude the presence of other unstated features, elements, components, groups, integers and/or steps as well as those that do not materially affect the basic and novel characteristic(s) and/or function of the claimed invention.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

### REFERENCES

1. Garside, M. U.S. Chemical industry-Statistics & Facts; Statista: 2020.
2. Sholl, D. S.; Lively, R. P., Seven chemical separations to change the world. Nature 2016, 532 (7600), 435-7.
3. Murnen, H. Separation Processes with Hope for Purification Energy Efficiency. www.chemical-materials.elsevier.com/chemical-manufacturing-excellence/separation-processes-for-energy-efficiency/.
4. Laboratory, O. R. N. Materials for Separation Technologies: Energy and Emission Reduction Opportunities; 2005.
5. Lively, R. P.; Realff, M. J., On thermodynamic separation efficiency: Adsorption processes. AIChE Journal 2016, 62 (10), 3699-3705.
6. Shao, L., Grand Challenges in Emerging Separation Technologies. Frontiers in Environmental Chemistry 2020, 1.
7. Yang, R. T., Gas separation by adsorption processes. Butterworth-Heinemann: 2013.
8. Walton, K. S.; Sholl, D. S., Predicting multicomponent adsorption: 50 years of the ideal adsorbed solution theory. AIChE Journal 2015, 61 (9), 2757-2762.
9. Walton, K. S., 110th Anniversary: Commentary: Perspectives on Adsorption of Complex Mixtures. Industrial & Engineering Chemistry Research 2019, 58 (37), 17100-17105.
10. Tun, H.; Chen, C. C., Prediction of mixed-gas adsorption equilibria from pure component adsorption isotherms. AIChE Journal 2020, 66 (7).
11. Suwanayuen, S.; Danner, R. P., Vacancy solution theory of adsorption from gas mixtures. AIChE Journal 1980, 26 (1), 76-83.
12. Wang, Z.; Li, Y.; Guo, P.; Meng, W., Analyzing the Adaption of Different Adsorption Models for Describing the Shale Gas Adsorption Law. Chemical Engineering & Technology 2016, 39 (10), 1921-1932.
13. Markham, E. C.; Benton, A. F., The Adsorption of Gas Mixtures by Silica. Journal of the American Chemical Society 1931, 53 (2), 497-507.
14. Myers, A. L.; Prausnitz, J. M., Thermodynamics of mixed-gas adsorption. AIChE Journal 1965, 11 (1), 121-127.
15. Sorial, G. A.; Granville, W. H.; Daly, W. O., Adsorption equilibria for oxygen and nitrogen gas mixtures on 5 A molecular sieves. Chemical Engineering Science 1983, 38 (9), 1517-1523.
16. Do, D. D., Adsorption Analysis: Equilibria and Kinetics. Imperial college press London: 1998; Vol. 2.
17. Wu, Q.; Zhou, L.; Wu, J.; Zhou, Y., Adsorption Equilibrium of the Mixture CH₄+N₂+H₂ on Activated Carbon. Journal of Chemical & Engineering Data 2005, 50 (2), 635-642.
18. Tien, C., Introduction to adsorption: Basics, analysis, and applications. Elsevier: 2018.
19. Hermann, M.; Niessen, W.; Karge, H. G., Sorption Kinetics of n-Paraffins in MFI-type Zeolites Investigated by micro-FTIR. In Fundamentals of Adsorption: Proceedings of the Fifth International Conference on Fundamentals of Adsorption, LeVan, M. D., Ed. Springer US: Boston, MA, 1996; pp 377-384.
20. Harlick, P. J. E.; Tezel, F. H., Adsorption of carbon dioxide, methane and nitrogen: pure and binary mixture adsorption for ZSM-5 with SiO₂/Al₂O₃ ratio of 280. Separation and Purification Technology 2003, 33 (2), 199-210.
21. Kaur, H.; Tun, H.; Sees, M.; Chen, C.-C., Local composition activity coefficient model for mixed-gas adsorption equilibria. Adsorption 2019, 25 (5), 951-964.
22. Chang, C.-K.; Tun, H.; Chen, C.-C., An activity-based formulation for Langmuir adsorption isotherm. Adsorption 2019, 26 (3), 375-386.
23. Tun, H.; Chen, C.-C., Isosteric heat of adsorption from thermodynamic Langmuir isotherm. Adsorption 2021.
24. Talu, O.; Li, J.; Myers, A. L., Activity coefficients of adsorbed mixtures. Adsorption 1995, 1 (2), 103-112.
25. Talu, O.; Myers, A. L., Rigorous thermodynamic treatment of gas adsorption. AIChE Journal 1988, 34 (11), 1887-1893.
26. Sing, K. S. W., Assessment of Surface Area by Gas Adsorption. In Adsorption by Powders and Porous Solids, Rouquerol, F.; Rouquerol, J.; Sing, K. S. W.; Llewellyn, P.; Maurin, G., Eds. Academic Press: Oxford, 2014; pp 237-268.
27. Britt, H. I.; Luecke, R. H., The Estimation of Parameters in Nonlinear, Implicit Models. Technometrics 1973, 15 (2), 233-247.
28. Costa, E.; Calleja, G.; Marron, C.; Jimenez, A.; Pau, J., Equilibrium adsorption of methane, ethane, ethylene, and propylene and their mixtures on activated carbon. Journal of Chemical & Engineering Data 1989, 34 (2), 156-160.
29. Wu, C.-W.; Kothare, M. V.; Sircar, S., Equilibrium Adsorption Isotherms of Pure N₂ and O₂ and Their Binary Mixtures on LiLSX Zeolite: Experimental Data and Thermodynamic Analysis. Industrial & Engineering Chemistry Research 2014, 53 (17), 7195-7201.
30. Talu, O.; Zwiebel, I., Multicomponent adsorption equilibria of nonideal mixtures. AIChE Journal 1986, 32 (8), 1263-1276.
31. Costa, E.; Sotelo, J. L.; Calleja, G.; Marrón, C., Adsorption of binary and ternary hydrocarbon gas mixtures on activated carbon: Experimental determination and theoretical prediction of the ternary equilibrium data. AIChE Journal 1981, 27 (1), 5-12.
32. Bezus, A.; Dreving, V.; Kiselev, A., Isotherms and heats of adsorption of propane and propylene on graphitized carbon black-energy of adsorption forces. Kolloidnyi Zhurnal 1961, 23 (4), 389-&.
33. Livingston, H., The cross-sectional areas of molecules adsorbed on solid surfaces. Journal of colloid science 1949, 4 (5), 447-458.
34. Cintas, E. N. Technoeconomic assessment of thermochemical biorefinery based on DME as a platform chemical. University of Seville, 2016.
35. Harris, B. L.; Emmett, P., Adsorption Studies. Physical Adsorption of Nitrogen, Toluene, Benzene, Ethyl Iodide, Hydrogen Sulfide, Water Vapor, Carbon Disulfide, and Pentane on Various Porous and Non-Porous Solids. The Journal of Physical Chemistry 1949, 53 (6), 811-825.
36. Renon, H.; Prausnitz, J. M., Local compositions in thermodynamic excess functions for liquid mixtures. AIChE Journal 1968, 14 (1), 135-144.
37. Scott, R. L., Corresponding States Treatment of Nonelectrolyte Solutions. The Journal of Chemical Physics 1956, 25 (2), 193-205.

What is claimed is:

1. A computerized method for estimating an adsorption equilibria for one or more gases from pure component adsorption isotherms comprising:
providing one or more processors, a memory communicably coupled to the one or more processors and an output device communicably coupled to the one or more processors;
calculating, using the one or more processors, an adsorption of each gas i on a constant monolayer adsorption surface A^{o} using generalized Langmuir isotherm equations:
$${θ}_{i} = \frac{{n}_{i} {A}_{i}}{{A}^{o}} = \frac{{K}_{i}^{o} {y}_{i} P}{\frac{{γ}_{i}}{{γ}_{ϕ} {q}_{i}} + {\sum }_{j = 1}^{n} \frac{{γ}_{i} {q}_{j}}{{γ}_{j} {q}_{i}} {K}_{j}^{o} {y}_{j} P}$$
$${θ}_{ϕ} = \frac{{n}_{ϕ} {A}_{ϕ}}{{A}^{o}} = \frac{1}{1 + {\sum }_{j = 1}^{n} \frac{{γ}_{ϕ} {q}_{j}}{{γ}_{j}} {K}_{j}^{o} {y}_{j} P}$$
$${q}_{i} = \frac{{A}_{i}}{{A}_{ϕ}}$$
where: θᵢ is an adsorbate phase area fraction covered with the gas i,
nᵢAᵢ is an occupied area for the gas i,
Kᵢ^{o} is an intrinsic adsorption equilibrium constant of the gas i,
yᵢ is a gas phase mole fraction of gas i,
P is a gas vapor pressure,
γᵢ is an activity coefficient of the gas i,
γ_{ϕ} is an activity coefficient of vacant sites,
qᵢ is a ratio of an effective area of the gas i (Aᵢ) and an effective area of a phantom molecule ϕ (A_{ϕ}),
n is a number of the one or more gases,
θ_{ϕ} is an adsorbate phase vacant site area fraction, and
n_{ϕ}A_{ϕ} is a vacant area for the phantom molecule ϕ;
providing the adsorption of each gas i to the output device; and
developing a chemical process or a product using the adsorption of each gas i.

2. The method of claim 1, wherein the generalized Langmuir isotherm equations reduce to
$$\frac{{n}_{i}}{{n}_{i}^{0}} = \frac{{K}_{i}^{o} {y}_{i} P}{1 + {\sum }_{i = 1}^{n} {K}_{i}^{o} {y}_{i} P}$$
when (1) the adsorbate and vacant site effective areas are the same A₁=A₂= . . . =Aᵢ=A_{ϕ}, or equivalently, the saturation loadings of adsorbates and phantom molecule are same n₁⁰=n₂⁰= . . . =nᵢ⁰=n_{ϕ}⁰, and (2) the adsorbate phase activity coefficients are unity γᵢ=γ_{ϕ}=1.

3. The method of claim 1, wherein the one or more gases comprise a mixed gas having two or more components.

4. The method of claim 1, wherein the constant monolayer adsorption surface comprises activated carbon, LiLSX or Zeolite H-mordenite.

5. The method of claim 1, wherein the gas i comprises CH₄, C₂H₄, C₂H₆, C₃H₆, N₂, O₂, CO₂, H₂S, or C₃H₈.

6. The method of claim 1, wherein the one or more gasses comprise a mixed gas selected from CH₄—C₂H₄, CH₄—C₂H₆, C₂H₄—C₂H₆, C₂H₄—C₃H₆, and C₂H₆—C₃H₆.

7. The method of claim 1, wherein the one or more gasses comprise a mixed gas selected from N₂ and O₂.

8. The method of claim 1, wherein the one or more gasses comprise a mixed gas selected from H₂S—CO₂, C₃H₈—H₂S, and C₃H₈—CO₂.

9. The chemical process or the product developed in accordance with claim 1.

10. A system for estimating an adsorption equilibria for one or more gases from pure component adsorption isotherms comprising:
at least one input/output interface;
a data storage;
one or more processors communicably coupled to the at least one input/output interface and the data storage, wherein the one or more processors calculate an adsorption of each gas i on a constant monolayer adsorption surface A^{o} using generalized Langmuir isotherm equations:
$${θ}_{i} = \frac{{n}_{i} {A}_{i}}{{A}^{o}} = \frac{{K}_{i}^{o} {y}_{i} P}{\frac{{γ}_{i}}{{γ}_{ϕ} {q}_{i}} + {\sum }_{j = 1}^{n} \frac{{γ}_{i} {q}_{j}}{{γ}_{j} {q}_{i}} {K}_{j}^{o} {y}_{j} P}$$
$${θ}_{ϕ} = \frac{{n}_{ϕ} {A}_{ϕ}}{{A}^{o}} = \frac{1}{1 + {\sum }_{j = 1}^{n} \frac{{γ}_{ϕ} {q}_{j}}{{γ}_{j}} {K}_{j}^{o} {y}_{j} P}$$
$${q}_{i} = \frac{{A}_{i}}{{A}_{ϕ}}$$
where: θᵢ is an adsorbate phase area fraction covered with the gas i,
nᵢAᵢ is an occupied area for the gas i,
Kᵢ^{o} is an intrinsic adsorption equilibrium constant of the gas i,
yᵢ is a gas phase mole fraction of gas i,
P is a gas vapor pressure,
γᵢ is an activity coefficient of the gas i,
γ_{ϕ} is an activity coefficient of vacant sites,
qᵢ is a ratio of an effective area of the gas i (Aᵢ) and an effective area of a phantom molecule ϕ (A_{ϕ}),
n is a number of the one or more gases,
θ_{ϕ} is an adsorbate phase vacant site area fraction, and
n_{ϕ}A_{ϕ} is a vacant area for the phantom molecule ϕ; and
wherein the adsorption of each gas i is provided to the output device, and a chemical process or a product is developed using the adsorption of each gas i.

11. The system of claim 10, wherein the generalized Langmuir isotherm equations reduce to
$$\frac{{n}_{i}}{{n}_{i}^{0}} = \frac{{K}_{i}^{o} {y}_{i} P}{1 + {\sum }_{i = 1}^{n} {K}_{i}^{o} {y}_{i} P}$$
when (1) the adsorbate and vacant site effective areas are the same A₁=A₂= . . . =Aᵢ=A_{ϕ}, or equivalently, the saturation loadings of adsorbates and phantom molecule are same n₁⁰=n₂⁰= . . . =nᵢ⁰=n_{ϕ}⁰, and (2) the adsorbate phase activity coefficients are unity γᵢ=γ_{ϕ}=1.

12. The system of claim 10, wherein the one or more gases comprise a mixed gas having two or more components.

13. The system of claim 10, wherein the constant monolayer adsorption surface comprises activated carbon, LiLSX or Zeolite H-mordenite.

14. The system of claim 10, wherein the gas i comprises CH₄, C₂H₄, C₂H₆, C₃H₆, N₂, O₂, CO₂, H₂S, or C₃H₈.

15. The system of claim 10, wherein the one or more gasses comprise a mixed gas selected from CH₄—C₂H₄, CH₄—C₂H₆, C₂H₄—C₂H₆, C₂H₄—C₃H₆, and C₂H₆—C₃H₆.

16. The system of claim 10, wherein the one or more gasses comprise a mixed gas selected from N₂ and O₂.

17. The system of claim 10, wherein the one or more gasses comprise a mixed gas selected from H₂S—C₂, C₃H₈—H₂S, and C₃H₈—CO₂.

18. A computer program embodied on a non-transitory computer readable storage medium that is executed using one or more processors for estimating an adsorption equilibria for one or more gases from pure component adsorption isotherms comprising:
a code segment for calculate an adsorption of each gas i on a constant monolayer adsorption surface A^{o} using generalized Langmuir isotherm equations:
$${θ}_{i} = \frac{{n}_{i} {A}_{i}}{{A}^{o}} = \frac{{K}_{i}^{o} {y}_{i} P}{\frac{{γ}_{i}}{{γ}_{ϕ} {q}_{i}} + {\sum }_{j = 1}^{n} \frac{{γ}_{i} {q}_{j}}{{γ}_{j} {q}_{i}} {K}_{j}^{o} {y}_{j} P}$$
$${θ}_{ϕ} = \frac{{n}_{ϕ} {A}_{ϕ}}{{A}^{o}} = \frac{1}{1 + {\sum }_{j = 1}^{n} \frac{{γ}_{ϕ} {q}_{j}}{{γ}_{j}} {K}_{j}^{o} {y}_{j} P}$$
$${q}_{i} = \frac{{A}_{i}}{{A}_{ϕ}}$$
where: θᵢ is an adsorbate phase area fraction covered with the gas i,
nᵢAᵢ is an occupied area for the gas i,
Kᵢ^{o} is an intrinsic adsorption equilibrium constant of the gas i,
yᵢ is a gas phase mole fraction of gas i,
P is a gas vapor pressure,
γᵢ is an activity coefficient of the gas i,
γ_{ϕ} is an activity coefficient of vacant sites,
qᵢ is a ratio of an effective area of the gas i (Aᵢ) and an effective area of a phantom molecule ϕ (A_{ϕ}),
n is a number of the one or more gases,
θ_{ϕ} is an adsorbate phase vacant site area fraction, and
n_{ϕ}A_{ϕ} is a vacant area for the phantom molecule ϕ; and
a code segment for developing a chemical process or a product using the adsorption of each gas i.

19. A method of adsorbing one or more gases comprising:
providing a vessel containing a constant monolayer adsorption surface A^{o};
introducing the one or more gasses into the vessel; and
wherein the adsorption of each gas i on the constant monolayer adsorption surface A^{o} is determined by generalized Langmuir isotherm equations:
$${θ}_{i} = \frac{{n}_{i} {A}_{i}}{{A}^{o}} = \frac{{K}_{i}^{o} {y}_{i} P}{\frac{{γ}_{i}}{{γ}_{ϕ} {q}_{i}} + {\sum }_{j = 1}^{n} \frac{{γ}_{i} {q}_{j}}{{γ}_{j} {q}_{i}} {K}_{j}^{o} {y}_{j} P}$$
$${θ}_{ϕ} = \frac{{n}_{ϕ} {A}_{ϕ}}{{A}^{o}} = \frac{1}{1 + {\sum }_{j = 1}^{n} \frac{{γ}_{ϕ} {q}_{j}}{{γ}_{j}} {K}_{j}^{o} {y}_{j} P}$$
$${q}_{i} = \frac{{A}_{i}}{{A}_{ϕ}}$$
where: θᵢ is an adsorbate phase area fraction covered with the gas i,
nᵢAᵢ is an occupied area for the gas i,
Kᵢ^{o} is an intrinsic adsorption equilibrium constant of the gas i,
yᵢ is a gas phase mole fraction of gas i,
P is a gas vapor pressure,
γᵢ is an activity coefficient of the gas i,
γ_{ϕ} is an activity coefficient of vacant sites,
qᵢ is a ratio of an effective area of the gas i (Aᵢ) and an effective area of a phantom molecule ϕ (A_{ϕ}),
n is a number of the one or more gases,
θ_{ϕ} is an adsorbate phase vacant site area fraction, and
n_{ϕ}A_{ϕ} is a vacant area for the phantom molecule ϕ.

20. The method of claim 19, wherein the generalized Langmuir isotherm equations reduce to
$$\frac{{n}_{i}}{{n}_{i}^{0}} = \frac{{K}_{i}^{o} {y}_{i} P}{1 + {\sum }_{i = 1}^{n} {K}_{i}^{o} {y}_{i} P}$$
when (1) the adsorbate and vacant site effective areas are the same A₁=A₂= . . . =Aᵢ=A_{ϕ}, or equivalently, the saturation loadings of adsorbates and phantom molecule are same n₁⁰=n₂⁰= . . . =nᵢ⁰=n_{ϕ}⁰, and (2) the adsorbate phase activity coefficients are unity γᵢ=γ_{ϕ}=1.

21. The method of claim 19, wherein the one or more gases comprise a mixed gas having two or more components.

22. The method of claim 19, wherein the constant monolayer adsorption surface comprises activated carbon, LiLSX or Zeolite H-mordenite.

23. The method of claim 19, wherein the gas i comprises CH₄, C₂H₄, C₂H₆, C₃H₆, N₂, O₂, CO₂, H₂S, or C₃H₈.

24. The method of claim 19, wherein the one or more gasses comprise a mixed gas selected from CH₄—C₂H₄, CH₄—C₂H₆, C₂H₄—C₂H₆, C₂H₄—C₃H₆, and C₂H₆—C₃H₆.

25. The method of claim 19, wherein the one or more gasses comprise a mixed gas selected from N₂ and O₂.

26. The method of claim 19, wherein the one or more gasses comprise a mixed gas selected from H₂S—C₂, C₃H₈—H₂S, and C₃H₈—CO₂.

27. A product produced in accordance with claim 19.
